(19) United States
(12) Patent Application Publication
(10) Pub. No.: US 2021/0214737 A1
(43) Pub. Date: Jul. 15, 2021

(54) SEED-SPECIFIC AND EMBRYO-PREFERENTIAL PROMOTERS AND USES THEREOF

(71) Applicant: BAYER CROPSCIENCE NV, Diegem (BE)

(72) Inventors: Peter DENOLF, Velzeke (BE); Katrien VAN AUDENHOVE, Gent (BE)

(21) Appl. No.: 16/093,572

(22) PCT Filed: Apr. 7, 2017
(86) PCT No.: PCT/EP2017/058390

§ 371 Date: Oct. 12, 2018

(30) Foreign Application Priority Data
Apr. 13, 2016 (EP) EP16165103.9

(51) Int. Cl. C12N 15/82

(52) U.S. Cl. CPC C12N 15/8234

(57) ABSTRACT

The present invention relates to Brassica sequences comprising late stage seed-specific and embryo-preferential promoter activity. Provided are recombinant genes comprising the late stage seed-specific and embryo-preferential promoter operably linked to a heterologous nucleic acid sequence, and cells, plants and seeds comprising the recombinant gene. The promoters can be used to alter gene expression specifically in the seeds at late developmental stages and preferentially in the embryo and to alter biotic or abiotic stress tolerance, yield, seed quality or seed properties.

### FIELD OF THE INVENTION

The present invention relates to materials and methods for the expression of a gene of interest specifically in seeds of plants. In particular, the invention provides an expression cassette for regulating seed-specific and embryo-preferential expression in plants.

### BACKGROUND

Modification of plants to alter and/or improve phenotypic characteristics (such as productivity or quality) requires the overexpression or down-regulation of endogenous genes or the expression of heterologous genes in plant tissues. Such genetic modification relies on the availability of a means to drive and to control gene expression as required. Indeed, genetic modification relies on the availability and use of suitable promoters which are effective in plants and which regulate gene expression so as to give the desired effect(s) in the transgenic plant.

For numerous applications in plant biotechnology a tissue-specific or a tissue-preferential expression profile is advantageous, since beneficial effects of expression in one tissue may have disadvantages in others.

Seed-preferential or seed-specific promoters are useful for expressing or down-regulating genes specifically in the seeds to get the desired function or effect, such as improving disease resistance, herbicide resistance, modifying seed or grain composition or quality, such as modifying starch quality or quantity, modifying oil quality or quantity, modifying amino-acid or protein composition, improving tolerance to biotic or abiotic stress, increasing yield, or altering metabolic pathways in the seeds.

Examples of seed-preferential or seed-specific promoters include the Tonoplast Intrinsic Protein alpha promoter from Arabidopsis thaliana (US patent application US2009/0241230), the KNAT411 promoter from Arabidopsis thaliana (U.S. Pat. No. 6,342,657), an oleosin promoter, a 2S storage protein promoter or a legumin-like seed storage protein promoter from Linum usitatissimum (U.S. Pat. No. 7,642,346), the acyl carrier protein promoter from Brassica napus (US Pat. Application No. 1994/0129129), the 3-amylase promoter of barley (US Pat. Application No. 1997/0793599), and the Ha ds10 G1 promoter of sunflower (U.S. Pat. No. 6,759,570).

There remains thus an interest in the isolation of novel late stage seed-specific promoters having embryo-preferential activity. It is thus an objective of the present invention to provide Brassica promoters having late stage seed-specific activity and embryo-preferential activity. This objective is solved by the present invention as herein further explained.

### SUMMARY

In one aspect, the invention provides an isolated nucleic acid comprising late stage seed-specific and embryo-preferential promoter activity selected from the group consisting of (a) a nucleic acid comprising a nucleotide sequence of any one of SEQ ID NOs: 2 to 10 or a functional fragment thereof; and (b) a nucleic acid comprising a nucleotide sequence having at least 80% sequence identity to any one of SEQ ID NOs: 2 to 10 or a functional fragment thereof.

A further embodiment provides a recombinant gene comprising the nucleic acid according to the invention operably linked to a heterologous nucleic acid sequence encoding an expression product of interest, and optionally a transcription termination and polyadenylation sequence, preferably a transcription termination and polyadenylation region functional in plant cells. In a further embodiment, said expression product of interest is an RNA capable of modulating the expression of a gene or is a protein.

Yet another embodiment provides a host cell, such as an E. coli cell, an Agrobacterium cell, a yeast cell, or a plant cell, comprising the isolated nucleic acid according to the invention, or the recombinant gene according to the invention.

In a further embodiment, a plant is provided comprising the recombinant gene according to the invention.

Yet a further embodiment provides seeds obtainable from the plant according to the invention. In another embodiment, the plants or plant parts according to the invention are seed crop plants or seeds.

Yet another embodiment provides a method of producing a transgenic plant comprising the steps of (a) introducing or providing the recombinant gene according to the invention to a plant cell to create transgenic cells; and (b) regenerating transgenic plants from said transgenic cell.

Further provided is a method of effecting late stage seed-specific and embryo-preferential expression of a nucleic acid comprising introducing the recombinant gene according to the invention into the genome of a plant, or providing the plant according to the invention. Also provided is a method for altering seed properties of a plant or to produce a commercially relevant product in a plant, said method comprising introducing the recombinant gene according to the invention into the genome of a plant, or providing the plant according to the invention. In another embodiment, said plant is a seed crop plant.

Also provided is the use of the isolated nucleic acid according to the invention to regulate expression of an operably linked nucleic acid in a plant, and the use of the isolated nucleic acid according to the invention, or the recombinant gene according to the invention to alter seed properties of a plant or to produce a commercially relevant product in a plant. In a further embodiment, said plant is a seed crop plant.

Yet another embodiment provides a method of producing food, feed, or an industrial product comprising (a) obtaining the plant or a part thereof, according to the invention; and (b) preparing the food, feed or industrial product from the plant or part thereof. In another embodiment, said food or feed is oil, meal, grain, starch, flour or protein, or said industrial product is biofuel, fiber, industrial chemicals, a pharmaceutical or a nutraceutical.

### BRIEF DESCRIPTION OF THE DRAWINGS

FIG. 1: GUS staining in the embryos carrying PcruP2 BnA2::GUS. A: GUS labelling in whole embryos at different developmental stages: sub-panel A: 10 to 12 DAF, sub-panel B: 15 to 18 DAF, sub-panel C: 20 to 23 DAF; sub-panel D: 23 to 27 DAF; sub-panel E: 27 to 32 DAF; sub-panel F: 35 to 40 DAF. B: GUS labelling in sectioned embryo radicle at early stage. C: GUS labelling in sectioned embryo at the mature embryo stage. Cot: cotyledon, vs: vasculature. D: Semi quantitative assessment of GUS labelling (μU*mg⁻¹ fresh weight*h) in transgenic lines carrying a PcruP2 BnA2::GUS T-DNA. Embryos stained at the following stages: 2: 13 to 15 DAF, 3: 16 to 18 DAF, 4: 19 to 23 DAF, 5: 25 to 29 DAF, 6: 31 to 35 DAF, 7: 37 to 40 DAF.

FIG. 2: Expression profile analysis in seeds carrying PcruP2 BnA2::GUS. A: GUS labelling in the endosperm and seed coat at late stage. The arrow indicates the endosperm. B: Semi quantitative assessment of GUS labelling (μU*mg⁻¹ fresh weight*h) in seed coats of seeds from transgenic lines carrying a PcruP2 BnA2::GUS T-DNA. Seed coats stained at the following stages: 2: 13 to 15 DAF, 3: 16 to 18 DAF, 4: 19 to 23 DAF, 5: 25 to 29 DAF, 6: 31 to 35 DAF.

FIG. 3: Alignment of the amino acid sequence of different Brassica CRUP2 proteins. Amino acid residues conserved in all proteins are indicated by an asterisk, conserved amino acid substitutions are indicated by a colon. The lowest identity between any two CRUP2 proteins is about 83%.

FIG. 4: Relative expression levels of different CRUP2 transcripts in different plant tissues. A: CRUP2 BnA2; B: CRUP2 BnA1; C: CRUP2 BnC1; D: CRUP2 Br2; E: CRUP2 Br1; F: CRUP2 Bo; G: CRUP2 BjA2; H: CRUP2 BjA1 and I: CRUP2 BjB1. Different tissues for A to F: AM33: Apical meristem 33 days after sowing (DAS); BFB42: Big flower buds 42 DAS; CTYL10: Cotyledons 10 DAS; OF52: Open flowers 52 DAS; Pod2: Pods 14-20 DAS; Pod3: Pods 21-25 DAS; Ro2w: Roots 14 DAS; SFB42: Small flower buds 42 DAS; Seed2: Seeds 14-20 days after flowering (DAF); Seed3: Seeds 21-25 DAF; Seed4: Seeds 26-30 DAF; Seed5: Seeds 31-35 DAF; Seed6: Seeds 42 DAF; Seed7: Seeds 49 DAF; St2w: Stem 14 DAS; St5w: Stem 33 DAS; YL33: Young leaf 33 DAS. Different tissues for G to I: AM22: Apical meristem 22 days after sowing (DAS); BFB35: Big flower buds 35 DAS; CTYL8: Cotyledons 8 DAS; OF35: Open flowers 35 DAS; Pod2: Pods 14-20 DAS; Pod3: Pods 21-25 DAS; Pod4: Pods 26-30 DAS; Pod5: Pods 31-35 DAS; Ro2W: Roots 14 DAS; SFB35: Small flower buds 35 DAS; Seed2: Seeds 14-20 days after flowering (DAF); Seed3: Seeds 21-25 DAF; Seed4: Seeds 26-30 DAF; Seed5: Seeds 31-35 DAF; Seed6: Seeds 42 DAF; Seed7: Seeds 49 DAF; St2w: Stem 14 DAS; St3w: Stem 22 DAS; YL22: Young leaf 22 DAS; OL22: Old leaf 22 DAS.

FIG. 5: Relative expression levels of different CRUP2 transcripts in different seed sub-tissues. A: CRUP2 BnA2; B: CRUP2 BnA1; C: CRUP2 BnC1; D: CRUP2 Br2; E: CRUP2 Br1 and F: CRUP2 Bo. Different seed sub-tissues: a: Endosperm, 18 days after flowering (DAF); b: Endosperm, 24 DAF; c: embryonic hypocotyl, 18 DAF; d: embryonic hypocotyl, 24 DAF; e: embryonic hypocotyl, 28 DAF; f: embryonic hypocotyl, 32 DAF; g: embryonic hypocotyl, 46 DAF; h: embryonic inner cotyledon, 18 DAF; i: embryonic inner cotyledon, 24 DAF; j: embryonic inner cotyledon, 28 DAF; k: embryonic inner cotyledon, 32 DAF; l: embryonic inner cotyledon, 46 DAF; m: embryonic outer cotyledon, 18 DAF; n: embryonic outer cotyledon(inner part), 24 DAF; o: embryonic outer cotyledon(inner part), 28 DAF; p: embryonic outer cotyledon(inner part), 32 DAF; q: embryonic outer cotyledon(inner part), 46 DAF; r: embryonic outer cotyledon(outer part), 24 DAF; s: embryonic outer cotyledon(outer part), 28 DAF; t: embryonic outer cotyledon(outer part), 32 DAF; u: embryonic outer cotyledon(outer part), 46 DAF.

FIG. 6: Alignment of the 3′ end of the nucleotide sequence of the Brassica PcruP2 promoters from Brassica napus, Brassica juncea, Brassica oleracea and Brassica rapa. The predicted TATA box is indicated by a frame. The transcription start is marked in bold. Conserved motifs are underlined and numbered. Motif 1 has the sequence of SEQ ID NO: 29, motif 2 has the sequence gtctaaya, motif 3 has the sequence of SEQ ID NO: 30, motif 4 has the sequence tcatcttaa, motif 5 has the sequence gakcarttc, motif 6 has the sequence of SEQ ID NO: 31, motif 7 has the sequence of SEQ ID NO: 32, motif 8 has the sequence of SEQ ID NO: 33, motif 9 has the sequence of SEQ ID NO: 34, motif 10 has the sequence of SEQ ID NO: 35, motif 11 has the sequence of SEQ ID NO: 36, motif 12 has the sequence of SEQ ID NO: 37, motif 13 has the sequence of SEQ ID NO: 38, motif 14 has the sequence of SEQ ID NO: 39, motif 15 has the sequence of SEQ ID NO: 40 and motif 16 has the sequence of SEQ ID NO: 41.

### DETAILED DESCRIPTION

The present invention is based on the observation that SEQ ID NOs: 2 to 10 have late stage seed-specific promoter activity and embryo-preferential promoter activity in Brassica.

SEQ ID NOs: 2 to 10 depict the region upstream (i.e. located 5′ upstream of) from the first ATG start codon of the CRUP2 BnA2, CRUP2 BnA1, CRUP2 BnC1, CRUP2 Br2, CRUP2 Br1, CRUP2 Bo, CRUP2 BjA2, CRUP2 BjA1, and CRUP2 BjB1 respectively.

CRUP2 BnA2, CRUP2 BnA1 and CRUP2 BnC1 are the 3 copies present in Brassica napus of the orthologous gene to the Arabidopsis thaliana Cruciferin 2 gene At1g03880. CRUP2 Bo is the one copy present in Brassica oleracea of the orthologous gene to the Arabidopsis thaliana Cruciferin 2 gene At1g03880. CRUP2 Br2 and CRUP2 Br1 are the 2 copies present in Brassica rapa of the orthologous gene to the Arabidopsis thaliana Cruciferin 2 gene At1g03880. CRUP2 BjA2, CRUP2 BjA1, CRUP2 BjB1 are the 3 copies present in Brassica juncea of the orthologous gene to the Arabidopsis thaliana cruciferin gene At1g03880. The cruciferin complex has an octameric structure (Nietzel et al. 2013). Sjodahl et al. 1993 studied the expression pattern of each cruciferin gene family in Brassica napus seed development. They concluded that the transcripts of the 3 families accumulate in the embryo axis and in the cotyledons. They showed that the transcript of the Cruciferin 2 gene family are absent from the root cap and the provascular cells but did not disclose the corresponding promoter sequence. Sjodahl et al. 1995 characterized the promoter region of the Cruciferin 1 gene of Brassica napus and Bilodeau et al. 1994 characterized the promoter region of the Cruciferin 3 gene of Brassica napus.

In one aspect, the invention provides an isolated nucleic acid comprising late stage seed-specific and embryo-preferential promoter activity selected from the group consisting of (a) a nucleic acid comprising a nucleotide sequence of any one of SEQ ID NOs: 2 to 10 or a functional fragment thereof; and (b) a nucleic acid comprising a nucleotide sequence having at least 80% sequence identity to anyone of SEQ ID NOs: 2 to 10, or a functional fragment thereof.

The nucleic acid comprising the late stage seed-specific and embryo-preferential promoter activity according to the invention may also be comprised in a larger DNA molecule.

“Seed-specific promoter activity” in the context of this invention means the promoter activity is at least 10 times, or at least 20 times, or at least 50 times, or at least 100 times, or at least 200 times, or at least 500 times, or even at least 1000 times higher in seeds than in other tissues. In other words, in seed-specific promoter activity, transcription of the nucleic acid operably linked to the promoter of the invention in the seeds is at least 10 times, or at least 20 times, or at least 50 times, or at least 100 times, or at least 200 times, or at least 500 times or even at least 1000 times higher than in other tissues. In other words, the seed-specific promoter drives seed-specific expression of the nucleic acid operably linked to the seed-specific promoter.

“Late stage seed” development in the context of this invention refers to seeds in which the embryo developmental stage ranges from green cotyledon stage to the mature embryo stage. These developmental stages are reached from 26 days after flowering.

“Seed-specific promoter activity” encompasses “embryo-preferential promoter activity”.

“Embryo-preferential promoter activity” in the context of this invention means the promoter activity is at least 2 times, or at least 5 times, or at least 10 times, or at least 20 times or even at least 100 times higher in embryonic tissues than in other seed tissues. In other words, in embryo-preferential promoter activity, transcription of the nucleic acid operably linked to the promoter of the invention in the embryo is at least 2 times, or at least 5 times, or at least 10 times, or at least 20 times or even at least 100 times higher than in other seed tissues. In other words, the embryo-preferential promoter drives embryo-preferential expression of the nucleic acid operably linked to the embryo-preferential promoter.

The phrase “operably linked” refers to the functional spatial arrangement of two or more nucleic acid regions or nucleic acid sequences. For example, a promoter region may be positioned relative to a nucleic acid sequence such that transcription of a nucleic acid sequence is directed by the promoter region. Thus, a promoter region is “operably linked” to the nucleic acid sequence. “Functionally linked” is an equivalent term.

The phrases “DNA”, “DNA sequence,” “nucleic acid sequence,” “nucleic acid molecule” “nucleotide sequence” and “nucleic acid” refer to a physical structure comprising an orderly arrangement of nucleotides. The DNA sequence or nucleotide sequence may be contained within a larger nucleotide molecule, vector, or the like. In addition, the orderly arrangement of nucleic acids in these sequences may be depicted in the form of a sequence listing, figure, table, electronic medium, or the like.

As used herein, “promoter” means a region of DNA sequence that is essential for the initiation of transcription of DNA, resulting in the generation of an RNA molecule that is complementary to the transcribed DNA; this region may also be referred to as a “5′ regulatory region.” Promoters are usually located upstream of the coding sequence to be transcribed and have regions that act as binding sites for RNA polymerase II and other proteins such as transcription factors (trans-acting protein factors that regulate transcription) to initiate transcription of an operably linked gene. Promoters may themselves contain sub-elements (i.e. promoter motifs) such as cis-elements or enhancer domains that regulate the transcription of operably linked genes. The promoters of this invention may be altered to contain “enhancer DNA” to assist in elevating gene expression. As is known in the art, certain DNA elements can be used to enhance the transcription of DNA. These enhancers often are found 5′ to the start of transcription in a promoter that functions in eukaryotic cells, but can often be inserted upstream (5′) or downstream (3′) to the coding sequence. In some instances, these 5′ enhancer DNA elements are introns. Among the introns that are useful as enhancer DNA are the 5′ introns from the rice actin 1 gene (see U.S. Pat. No. 5,641,876), the rice actin 2 gene, the maize alcohol dehydrogenase gene, the maize heat shock protein 70 gene (see U.S. Pat. No. 5,593,874), the maize shrunken 1 gene, the light sensitive 1 gene of Solanum tuberosum, the Arabidopsis histon 4 intron and the heat shock protein 70 gene of Petunia hybrids (see U.S. Pat. No. 5,659,122). Thus, as contemplated herein, a promoter or promoter region includes variations of promoters derived by inserting or deleting regulatory regions, subjecting the promoter to random or site-directed mutagenesis, etc. The activity or strength of a promoter may be measured in terms of the amounts of RNA it produces, or the amount of protein accumulation in a cell or tissue, relative to a promoter whose transcriptional activity has been previously assessed or relative to a promoter driving the expression of a housekeeping gene.

A promoter as used herein may thus include sequences downstream of the transcription start, such as sequences coding the 5′ untranslated region (5′ UTR) of the RNA, introns located downstream of the transcription start, or even sequences encoding the protein. A functional promoter fragment according to the invention may comprise its own 5′UTR comprising the nucleotide sequence of SEQ ID NO: 2 from nucleotide 1495 to nucleotide 1543, or comprising the nucleotide sequence of any one of SEQ ID NOs: 3 to 9 from nucleotide 1452 to nucleotide 1500, or comprising the nucleotide sequence of SEQ ID NO: 10 from nucleotide 1451 to nucleotide 1500. Alternatively, 5′UTR fragments from other Brassica CRUP2 genes may be used. For example, a promoter fragment of SEQ ID NO: 2 may have the nucleotide sequence of said sequence from position 1495 to 1543 replaced by the nucleotide sequence of any one of SEQ ID NOs: 3 to 9 from nucleotide 1452 to nucleotide 1500. A promoter fragment of SEQ ID NO: 2 may have the nucleotide sequence of said sequence from position 1495 to 1543 replaced by the nucleotide sequence of SEQ ID NO: 10 from nucleotide 1451 to nucleotide 1500. A promoter fragment of any one of SEQ ID NOs: 3 to 9 may have the nucleotide sequence of said sequence from position 1452 to 1500 replaced by the nucleotide sequence of SEQ ID NO: 2 from nucleotide 1495 to nucleotide 1543. A promoter fragment of any one of SEQ ID NOs: 3 to 9 may have the nucleotide sequence of said sequence from position 1452 to 1500 replaced by the nucleotide sequence of any one of SEQ ID NOs: 3 to 9 from nucleotide 1452 to nucleotide 1500. As another example, a promoter fragment of any one of SEQ ID NOs: 3 to 9 may have the nucleotide sequence of said sequence from position 1452 to position 1500 replaced by the nucleotide sequence of SEQ ID NO: 10 from nucleotide 1451 to nucleotide 1500. A promoter fragment of SEQ ID NO: 10 may have the nucleotide sequence of said sequence from position 1451 to position 1500 replaced by the nucleotide sequence of SEQ ID NO: 2 from nucleotide 1495 to nucleotide 1543. A promoter fragment of SEQ ID NO: 10 may have the nucleotide sequence of said sequence from position 1451 to position 1500 replaced by the nucleotide sequence of any one of SEQ ID NOs: 3 to 9 from nucleotide 1452 to nucleotide 1500.

Such a promoter fragment may be at least about 500 bp, at least about 550 bp, at least about 600 bp, at least about 700 bp, at least about 800 bp, at least about 900 bp, at least about 1000 bp, at least about 1100 bp, at least about 1200 bp, at least about 1300 bp, at least about 1400 bp, at least about 1500 bp, or at least about 1550 bp upstream of the first ATG start codon of the CRUP2 transcripts and have late stage seed-specific and embryo-preferential promoter activity. In combination with the above described promoter fragments, a promoter fragment according to the invention may thus comprise the nucleotide sequence of SEQ ID NO: 2 from the nucleotide at position 1043 to the nucleotide at position 1543, the nucleotide sequence of SEQ ID NO: 2 from the nucleotide at position 993 to the nucleotide at position 1543, the nucleotide sequence of SEQ ID NO: 2 from the nucleotide at position 893 to the nucleotide at position 1543, the nucleotide sequence of SEQ ID NO: 2 from the nucleotide at position 793 to the nucleotide at position 1543, the nucleotide sequence of SEQ ID NO: 2 from the nucleotide at position 693 to the nucleotide at position 1543, the nucleotide sequence of SEQ ID NO: 2 from the nucleotide at position 593 to the nucleotide at position 1543, the nucleotide sequence of SEQ ID NO: 2 from the nucleotide at position 493 to the nucleotide at position 1543, the nucleotide sequence of SEQ ID NO: 2 from the nucleotide at position 393 to the nucleotide at position 1543, the nucleotide sequence of SEQ ID NO: 2 from the nucleotide at position 293 to the nucleotide at position 1543, the nucleotide sequence of SEQ ID NO: 2 from the nucleotide at position 193 to the nucleotide at position 1543, or the nucleotide sequence of SEQ ID NO: 2 from the nucleotide at position 93 to the nucleotide at position 1543. A promoter fragment according to the invention may also comprise the nucleotide sequence of SEQ ID NO: 5 from the nucleotide position 1000 to the nucleotide position 1500, the nucleotide sequence of SEQ ID NO: 5 from the nucleotide position 950 to the nucleotide position 1500, the nucleotide sequence of SEQ ID NO: 5 from the nucleotide position 900 to the nucleotide position 1500, the nucleotide sequence of SEQ ID NO: 5 from the nucleotide position 800 to the nucleotide position 1500, the nucleotide sequence of SEQ ID NO: 5 from the nucleotide position 700 to the nucleotide position 1500, the nucleotide sequence of SEQ ID NO: 5 from the nucleotide position 600 to the nucleotide position 1500, the nucleotide sequence of SEQ ID NO: 5 from the nucleotide position 500 to the nucleotide position 1500, the nucleotide sequence of SEQ ID NO: 5 from the nucleotide position 400 to the nucleotide position 1500, the nucleotide sequence of SEQ ID NO: 5 from the nucleotide position 300 to the nucleotide position 1500, the nucleotide sequence of SEQ ID NO: 5 from the nucleotide position 200 to the nucleotide position 1500, or the nucleotide sequence of SEQ ID NO: 5 from the nucleotide position 100 to the nucleotide position 1500. A promoter fragment according to the invention may also comprise the nucleotide sequence of any one of SEQ ID NOs: 3, 6 and 9 from the nucleotide position 950 to the nucleotide position 1500, the nucleotide sequence of any one of SEQ ID NOs: 3, 6 and 9 from the nucleotide position 900 to the nucleotide position 1500, the nucleotide sequence of any one of SEQ ID NOs: 3, 6 and 9 from the nucleotide position 800 to the nucleotide position 1500, the nucleotide sequence of any one of SEQ ID NOs: 3, 6 and 9 from the nucleotide position 700 to the nucleotide position 1500, the nucleotide sequence of any one of SEQ ID NOs: 3, 6 and 9 from the nucleotide position 600 to the nucleotide position 1500, the nucleotide sequence of any one of SEQ ID NOs: 3, 6 and 9 from the nucleotide position 500 to the nucleotide position 1500, the nucleotide sequence of any one of SEQ ID NOs: 3, 6 and 9 from the nucleotide position 400 to the nucleotide position 1500, the nucleotide sequence of any one of SEQ ID NOs: 3, 6 and 9 from the nucleotide position 300 to the nucleotide position 1500, the nucleotide sequence of any one of SEQ ID NOs: 3, 6 and 9 from the nucleotide position 200 to the nucleotide position 1500, or the nucleotide sequence of any one of SEQ ID NOs: 3, 6 and 9 from the nucleotide position 1000 to the nucleotide position 1500. A promoter fragment according to the invention may also comprise the nucleotide sequence of any one of SEQ ID NOs: 4, 7, 8 and 10 from the nucleotide position 900 to the nucleotide position 1500, the nucleotide sequence of any one of SEQ ID NOs: 4, 7, 8 and 10 from the nucleotide position 800 to the nucleotide position 1500, the nucleotide sequence of any one of SEQ ID NOs: 4, 7, 8 and 10 from the nucleotide position 700 to the nucleotide position 1500, the nucleotide sequence of any one of SEQ ID NOs: 4, 7, 8 and 10 from the nucleotide position 600 to the nucleotide position 1500, the nucleotide sequence of any one of SEQ ID NOs: 4, 7, 8 and 10 from the nucleotide position 500 to the nucleotide position 1500, the nucleotide sequence of any one of SEQ ID NOs: 4, 7, 8 and 10 from the nucleotide position 400 to the nucleotide position 1500, the nucleotide sequence of any one of SEQ ID NOs: 4, 7, 8 and 10 from the nucleotide position 300 to the nucleotide position 1500, the nucleotide sequence of any one of SEQ ID NOs: 4, 7, 8 and 10 from the nucleotide position 200 to the nucleotide position 1500, or the nucleotide sequence of any one of SEQ ID NOs: 4, 7, 8 and 10 from the nucleotide position 100 to the nucleotide position 1500.

Promoter activity for a functional promoter fragment in seeds may be determined by those skilled in the art, for example using analysis of RNA accumulation produced from the nucleic acid which is operably linked to the promoter as described herein, whereby the nucleic acid which is operably linked to the promoter can be the nucleic acid which is naturally linked to the promoter, i.e. the endogenous gene of which expression is driven by the promoter.

The late stage seed-specific expression capacity and the embryo-preferential expression capacity of the identified or generated fragments of the promoters of the invention can be conveniently tested by determining levels of the transcript of which expression is naturally driven by the promoter of the invention, i.e. endogenous transcript levels, such as, for example, using the methods as described herein in the Examples. Further, the late stage seed-specific and embryo-preferential expression capacity of the identified or generated fragments of the promoters of the invention can be conveniently tested by operably linking such DNA molecules to a nucleotide sequence encoding an easy scorable marker, e.g. a beta-glucuronidase gene, introducing such a chimeric gene into a plant and analyzing the expression pattern of the marker in seeds at different developmental stages as compared with the expression pattern of the marker in other parts of the plant or in seeds at other developmental stages. Other candidates for a marker (or a reporter gene) are chloramphenicol acetyl transferase (CAT) and proteins with fluorescent properties, such as green fluorescent protein (GFP) from Aequora victoria, or proteins with luminescent properties such as the Renilla luciferase or the bacterial lux operon. To define a minimal promoter region, a DNA segment representing the promoter region is removed from the 5′ region of the gene of interest and operably linked to the coding sequence of a marker (reporter) gene by recombinant DNA techniques well known to the art. The reporter gene is operably linked downstream of the promoter, so that transcripts initiating at the promoter proceed through the reporter gene. Reporter genes generally encode proteins, which are easily measured, including, but not limited to, chloramphenicol acetyl transferase (CAT), beta-glucuronidase (GUS), green fluorescent protein (GFP), beta-galactosidase (beta-GAL), and luciferase. The expression cassette containing the reporter gene under the control of the promoter can be introduced into an appropriate cell type by transfection techniques well known to the art. To assay for the reporter protein, cell lysates are prepared and appropriate assays, which are well known in the art, for the reporter protein are performed. For example, if CAT were the reporter gene of choice, the lysates from cells transfected with constructs containing CAT under the control of a promoter under study are mixed with isotopically labeled chloramphenicol and acetyl-coenzyme A (acetyl-CoA). The CAT enzyme transfers the acetyl group from acetyl-CoA to the 2- or 3-position of chloramphenicol. The reaction is monitored by thin-layer chromatography, which separates acetylated chloramphenicol from unreacted material. The reaction products are then visualized by autoradiography. The level of enzyme activity corresponds to the amount of enzyme that was made, which in turn reveals the level of expression and the late stage seed-specific and embryo-preferential functionality from the promoter or promoter fragment of interest. This level of expression can also be compared to other promoters to determine the relative strength of the promoter under study. Once activity and functionality is confirmed, additional mutational and/or deletion analyses may be employed to determine the minimal region and/or sequences required to initiate transcription. Thus, sequences can be deleted at the 5′ end of the promoter region and/or at the 3′ end of the promoter region, and nucleotide substitutions introduced. These constructs are then again introduced in cells and their activity and/or functionality determined.

The activity or strength of a promoter may be measured in terms of the amount of mRNA or protein accumulation it specifically produces, relative to the total amount of mRNA or protein. The promoter preferably expresses an operably linked nucleic acid sequence at a level greater than about 0.01%, about 0.02%, more preferably greater than about 0.05% of the total mRNA. Alternatively, the activity or strength of a promoter may be expressed relative to a well-characterized promoter (for which transcriptional activity was previously assessed).

It will herein further be clear that equivalent late stage seed-specific and embryo-preferential promoters can be isolated from other plants. To this end, equivalent promoters can be isolated using the coding sequences of the genes driven by the promoters of any one of SEQ ID NOs: 2 to 10 to screen a genomic library (e.g. by hybridization or in silico) of a crop of interest. When sufficient identity between the coding sequences is obtained (for example, higher than 80% identity) then promoter regions can be isolated upstream of the orthologous genes.

Suitable to the invention are nucleic acids comprising late stage seed-specific and embryo preferential promoter activity which comprise a nucleotide sequence having at least 40%, at least 50%, or at least 60%, or at least 70%, or at least 80%, or at least 85%, or at least 90%, or at least 95%, or at least 98% sequence identity to the herein described promoters and promoter regions or functional fragments thereof and are also referred to as variants. The term “variant” with respect to the transcription regulating nucleotide sequences SEQ ID NOs: 2 to 10 of the invention is intended to mean substantially similar sequences. Naturally occurring allelic variants such as these can be identified with the use of well-known molecular biology techniques, as, for example, with polymerase chain reaction (PCR) as herein outlined before. Variant nucleotide sequences also include synthetically derived nucleotide sequences, such as those generated, for example, by using site-directed mutagenesis of any one of SEQ ID NOs: 2 to 10. Generally, nucleotide sequence variants of the invention will have at least 40%, 50%, 60%, to 70%, e.g., preferably 71%, 72%, 73%, 74%, 75%, 76%, 77%, 78%, to 79%, generally at least 80%, e.g., 81% to 84%, at least 85%, e.g., 86%, 87%, 88%, 89%, 90%, 91%, 92%, 93%, 94%, 95%, 96%, 97%, to 98% and 99% nucleotide sequence identity to the native (wild type or endogenous) nucleotide sequence or a functional fragment thereof. Derivatives of the DNA molecules disclosed herein may include, but are not limited to, deletions of sequence, single or multiple point mutations, alterations at a particular restriction enzyme site, addition of functional elements, or other means of molecular modification which may enhance, or otherwise alter promoter expression. Techniques for obtaining such derivatives are well-known in the art (see, for example, J. F. Sambrook, D. W. Russell, and N. Irwin (2000) Molecular Cloning: A Laboratory Manual, 3^{rd} edition Volumes 1, 2, and 3. Cold Spring Harbor Laboratory Press). For example, one of ordinary skill in the art may delimit the functional elements within the promoters disclosed herein and delete any non-essential elements. Functional elements may be modified or combined to increase the utility or expression of the sequences of the invention for any particular application. Those of skill in the art are familiar with the standard resource materials that describe specific conditions and procedures for the construction, manipulation, and isolation of macromolecules (e.g., DNA molecules, plasmids, etc.), as well as the generation of recombinant organisms and the screening and isolation of DNA molecules. As used herein, the term “percent sequence identity” refers to the percentage of identical nucleotides between two segments of a window of optimally aligned DNA. Optimal alignment of sequences for aligning a comparison window are well-known to those skilled in the art and may be conducted by tools such as the local homology algorithm of Smith and Waterman (Waterman, M. S. Introduction to Computational Biology: Maps, sequences and genomes. Chapman & Hall. London (1995), the homology alignment algorithm of Needleman and Wunsch (J. Mol. Biol., 48:443-453 (1970), the search for similarity method of Pearson and Lipman (Proc. Natl. Acad. Sci., 85:2444 (1988), and preferably by computerized implementations of these algorithms such as GAP, BESTFIT, FASTA, and TFASTA available as part of the GCG (Registered Trade Mark), Wisconsin Package (Registered Trade Mark from Accelrys Inc., San Diego, Calif.). An “identity fraction” for aligned segments of a test sequence and a reference sequence is the number of identical components that are shared by the two aligned sequences divided by the total number of components in the reference sequence segment, i.e., the entire reference sequence or a smaller defined part of the reference sequence. Percent sequence identity is represented as the identity fraction times 100. The comparison of one or more DNA sequences may be to a full-length DNA sequence or a portion thereof, or to a longer DNA sequence.

A nucleic acid comprising a nucleotide sequence having at least 80% sequence identity to any one of SEQ ID NOs: 2 to 10 can thus be a nucleic acid comprising a nucleotide sequence having at least at least 80%, or at least 85%, or at least 90%, or at least 95%, or at least 98%, or 100% sequence identity to anyone of SEQ ID NOs: 2 to 10.

A “functional fragment” of a nucleic acid comprising late stage seed-specific and embryo-preferential promoter denotes a nucleic acid comprising a stretch of the nucleic acid sequences of any one of SEQ ID NOs: 2 to 10, or of the nucleic acid having at least 80% sequence identity to any one of SEQ ID NOs: 2 to 10 which still exerts the desired function, i.e. which has late stage seed-specific and embryo-preferential promoter activity. Assays for determining late stage seed-specific and embryo-preferential promoter activity are provided herein. Preferably, the functional fragment of the late stage seed-specific and embryo-preferential promoter contains the conserved promoter motifs, such as, for example, conserved promoter motifs as described in DoOP (doop.abc.hu, databases of Orthologous Promoters, Barta E. et al (2005) Nucleic Acids Research Vol. 33, D86-D90). A functional fragment may be a fragment of at least about 500 bp, at least about 550 bp, at least about 600 bp, at least about 700 bp, at least about 800 bp, at least about 900 bp, at least about 1000 bp, at least about 1100 bp, at least about 1200 bp, at least about 1300 bp, at least about 1400 bp, at least 1500 bp from the translation start site.

A nucleic acid comprising the nucleotide sequence of any one of SEQ ID NOs: 2 to 10 which further comprises insertion, deletion, substitution of at least 1 nucleotide up to 20 nucleotides, at least 1 nucleotide up to 15 nucleotides, at least 1 nucleotide up to 10 nucleotides, at least 1 nucleotide up to 5 nucleotides, at least 1 nucleotide up to 4 nucleotides, at least 1 nucleotide up to 3 nucleotides, or even at least 1 nucleotide up to 2 nucleotides may cover at least about 500 bp, at least about 550 bp, at least about 600 bp, at least about 700 bp, at least about 800 bp, at least about 900 bp, at least about 1000 bp, at least about 1100 bp, at least about 1200 bp, at least about 1300 bp, at least about 1400 bp, at least 1500 bp from the translation start site.

A number of consensus elements (sequence motifs) were identified on the promoter sequence disclosed herein.

Variants of the promoter described herein include those which comprise the identified motifs—motif 1 (SEQ ID NO: 29), motif 2 (gtctaaya), motif 3 (SEQ ID NO: 30), motif 4 (tcatcttaa), motif 5 (gakcarttc), motif 6 (SEQ ID NO: 31), motif 7 (SEQ ID NO: 32), motif 8 (SEQ ID NO: 33), motif 9 (SEQ ID NO: 34), motif 10 (SEQ ID NO: 35), motif 11 (SEQ ID NO: 36), motif 12 (SEQ ID NO: 37), motif 13 (SEQ ID NO: 38), motif 14 (SEQ ID NO: 39), motif 15 (SEQ ID NO: 40) and/or motif 16 (SEQ ID NO: 41)—but have otherwise been modified to delete nucleotide stretches within the sequence which are not needed for the promoter to be functional in seed-specific and embryo-preferential manner. For example, any nucleotide stretch located between the motifs and/or between the translational start and the first motif may be at least partially deleted to result in a shorter nucleotide sequence than the about 1500 bp sequence of anyone of SEQ ID NO: 2 to SEQ ID NO: 10.

A number of putative response elements were identified on the promoter sequence disclosed herein. The search was limited to seed-specific elements and two RY-repeat elements (CATGCA) were identified in all the promoters herein described. RY-repeat elements have been described as necessary for seed-specific expression in Brassica napus (Ezcurra et al. 1999).

Variants of the promoters described herein include those which comprise the identified RY-repeat elements, but have otherwise been modified to delete nucleotide stretches within the sequence which are not needed for the promoter to be function in a late stage seed-specific and embryo-preferential manner.

“Isolated nucleic acid”, used interchangeably with “isolated DNA” as used herein refers to a nucleic acid not occurring in its natural genomic context, irrespective of its length and sequence. Isolated DNA can, for example, refer to DNA which is physically separated from the genomic context, such as a fragment of genomic DNA. Isolated DNA can also be an artificially produced DNA, such as a chemically synthesized DNA, or such as DNA produced via amplification reactions, such as polymerase chain reaction (PCR) well-known in the art. Isolated DNA can further refer to DNA present in a context of DNA in which it does not occur naturally. For example, isolated DNA can refer to a piece of DNA present in a plasmid. Further, the isolated DNA can refer to a piece of DNA present in another chromosomal context than the context in which it occurs naturally, such as for example at another position in the genome than the natural position, in the genome of another species than the species in which it occurs naturally, or in an artificial chromosome.

A further embodiment provides a recombinant gene comprising the nucleic acid according to the invention operably linked to a heterologous nucleic acid sequence encoding an expression product of interest, and optionally a transcription termination and polyadenylation sequence, preferably a transcription termination and polyadenylation region functional in plant cells. In a further embodiment, said expression product of interest an RNA capable of modulating the expression of a gene or is a protein.

The term “expression product” refers to a product of transcription. Said expression product can be the transcribed RNA. It is understood that the RNA which is produced is a biologically active RNA. Said expression product can also be a peptide, a polypeptide, or a protein, when said biologically active RNA is an mRNA and said protein is produced by translation of said mRNA.

Alternatively, the heterologous nucleic acid, operably linked to the promoters of the invention, may also code for an RNA capable of modulating the expression of a gene. Said RNA capable of modulating the expression of a gene can be an RNA which reduces expression of a gene. Said RNA can reduce the expression of a gene for example through the mechanism of RNA-mediated gene silencing.

Said RNA capable of modulating the expression of a gene can be a silencing RNA down-regulating expression of a target gene. As used herein, “silencing RNA” or “silencing RNA molecule” refers to any RNA molecule, which upon introduction into a plant cell, reduces the expression of a target gene. Such silencing RNA may e.g. be so-called “antisense RNA”, whereby the RNA molecule comprises a sequence of at least 20 consecutive nucleotides having 95% sequence identity to the complement of the sequence of the target nucleic acid, preferably the coding sequence of the target gene. However, antisense RNA may also be directed to regulatory sequences of target genes, including the promoter sequences and transcription termination and polyadenylation signals. Silencing RNA further includes so-called “sense RNA” whereby the RNA molecule comprises a sequence of at least 20 consecutive nucleotides having 95% sequence identity to the sequence of the target nucleic acid. Other silencing RNA may be “unpolyadenylated RNA” comprising at least 20 consecutive nucleotides having 95% sequence identity to the complement of the sequence of the target nucleic acid, such as described in WO01/12824 or U.S. Pat. No. 6,423,885 (both documents herein incorporated by reference). Yet another type of silencing RNA is an RNA molecule as described in WO03/076619 (herein incorporated by reference) comprising at least 20 consecutive nucleotides having 95% sequence identity to the sequence of the target nucleic acid or the complement thereof, and further comprising a largely-double stranded region as described in WO03/076619 (including largely double stranded regions comprising a nuclear localization signal from a viroid of the Potato spindle tuber viroid-type or comprising CUG trinucleotide repeats). Silencing RNA may also be double stranded RNA comprising a sense and antisense strand as herein defined, wherein the sense and antisense strand are capable of base-pairing with each other to form a double stranded RNA region (preferably the said at least 20 consecutive nucleotides of the sense and antisense RNA are complementary to each other). The sense and antisense region may also be present within one RNA molecule such that a hairpin RNA (hpRNA) can be formed when the sense and antisense region form a double stranded RNA region. hpRNA is well-known within the art (see e.g WO99/53050, herein incorporated by reference). The hpRNA may be classified as long hpRNA, having long, sense and antisense regions which can be largely complementary, but need not be entirely complementary (typically larger than about 200 bp, ranging between 200 and 1000 bp). hpRNA can also be rather small ranging in size from about 30 to about 42 bp, but not much longer than 94 bp (see WO04/073390, herein incorporated by reference). Silencing RNA may also be artificial micro-RNA molecules as described e.g. in WO2005/052170, WO2005/047505 or US 2005/0144667, or ta-siRNAs as described in WO2006/074400 (all documents incorporated herein by reference). Said RNA capable of modulating the expression of a gene can also be an RNA ribozyme.

Said RNA capable of modulating the expression of a gene can modulate, preferably down-regulate, the expression of other genes (i.e. target genes) comprised within the seeds or even of genes present within a pathogen or pest that feeds upon the seeds of the transgenic plant such as a virus, fungus, insect, bacteria.

The nucleic acid sequence heterologous to the promoters according to the invention may generally be any nucleic acid sequence effecting increased, altered (e.g. in a different organ) or reduced level of transcription of a gene for which such expression modulation is desired. The nucleic acid sequence can for example encode a protein of interest. Exemplary genes for which an increased or reduced level of transcription may be desired in the seeds are e.g. nucleic acids that can provide an agriculturally or industrially important feature in seeds. Suitable heterologous nucleic acid sequences of interest include nucleic acids modulating expression of genes conferring resistance to diseases, stress tolerance genes, genes involved at different stages of fatty acid biosynthesis or degradation, in acyl editing, in storage compound storage or breakdown, genes encoding epoxidases, hydroxylases, cytochrome P450 mono-oxygenases, desaturases, tocopherol biosynthetic enzymes, carotenoid biosynthesis enzymes, amino acid biosynthetic enzymes, steroid pathway enzymes, starch branching enzymes, genes encoding proteins involved in starch synthesis, glycolysis, carbon metabolism, oxidative pentose phosphate cycle, protein synthesis, organelle organization and biogenesis, DNA metabolism, DNA replication, cell cycle, cell organization and biogenesis, cell proliferation, chromosome organization and biogenesis, microtubule-based processes, microtubule-based movement, cytoskeleton-dependent intracellular transport, cytoskeleton organization and biogenesis, chromatin assembly or disassembly, DNA-dependent DNA replication, chromosome organization and biogenesis, DNA packaging, establishment and/or maintenance of chromatin architecture, regulation of progression through the cell cycle, regulation of the cell cycle, nucleobase, nucleoside, nucleotide and nucleic acid metabolism, chromatin assembly, macromolecule biosynthesis, intracellular transport, establishment of cellular localization, cellular localization, nucleosome assembly, macromolecule metabolism, or M-phase; genes involved in secondary metabolism or genes involved in seed and/or seed coat architecture.

Genes involved in the fatty acid biosynthesis or degradation include but are not limited to genes encoding an acyl-CoA synthetase, a glycerol-phosphate acyltransferase, an O-acyltransferase, a lyso-phosphatidic acid acyltransferase, a phosphatidic acid phosphatase, a diacylglycerol acyltransferase, an oleate desaturases, a linoleate desaturases, an acyl-CoA hydroxylase, an acyl-lipid hydroxylase, a fatty acid epoxidase, a phospholipid:sterol acyltransferase, a phospholipid: diacylglycerol acyltransferase, a diacylglycerol transacylase, a lysophosphatidylcholine acyltransferase, a phosphatidylcholine:diacylglycerol cholinephosphotransferase, an acyl-CoA elongase, an acyl-lipid elongase, a phosphatidylglycerol-phosphate synthetase, a phosphatidylglycerol-phosphate phosphatase, a CDP-diacylglycerol synthetase, a phosphatidylinositol synthase, a phosphatidylserine synthase, a choline kinase, an ethanolamine kinase, a CDP-choline synthetase, a CDP-ethanolamine synthetase, a phosphatidylserine decarboxylase, a lipoxygenase, a phospholipase, a lipase, a carboxylesterase, a fatty alcohol reductase, a wax ester synthase, a bifunctional acyltranferases/wax synthase, a ketoacyl-CoA synthase, a ketoacyl-CoA reductase, a hydroxylacyl-CoA dehydrase, an enoyl-CoA reductase, an alcohol-forming fatty acyl-CoA reductase, an aldehyde-forming fatty acyl-CoA reductase, an aldehyde decarbonylase, a wax ester hydrolase, a glycerol-3-P-dehydrogenase, a CDP-choline:1,2-diacylglycerol cholinephosphotransferase, an oxidase, a ketosphinganine reductase, a ceramide synthase, an acylglycerophosphorylcholine acyltransferase, an acylglycerol-phosphate acyltransferase, a phosphoethanolamine N-methyltransferase, a ceramide sphingobase desaturase, a glucosylceramide synthase, a acyl-ceramide synthase, a triacylglycerol lipase, a monoacylglycerol lipase, an acyl-CoA oxidase, an hydroxyacyl-CoA dehydrogenase, a dienoyl-CoA reductase, a fatty acid omega-alcohol oxidase, a monoacylglycerol lipase, an acyl-CoA oxidase, a hydroxyacyl-CoA dehydrogenase, a dienoyl-CoA reductase, a fatty acid omega-alcohol oxidase, a fatty acid/acyl-CoA transporter, a acyl-CoA dehydrogenase, a diacylglycerol-phosphate kinase, a lysophosphatidic acic phosphatase, a peroxygenase; a Δ4-desaturase; a Δ5-desaturase, a Δ6-desaturase; a Δ9-desaturase, a Δ12-desaturase or a Δ15-desaturase.

Genes involved in cell proliferation include but are not limited to genes encoding Da1 (Li et al., 2008, Genes Dev 22:1331, WO2015/067943), Da2, EOD1 or EOD3 (WO2015/022192, PCT/GB2013/050072).

A “transcription termination and polyadenylation region” as used herein is a sequence that drives the cleavage of the nascent RNA, whereafter a poly(A) tail is added at the resulting RNA 3′ end, functional in plant cells. Transcription termination and polyadenylation signals functional in plant cells include, but are not limited to, 3′nos, 3′35S, 3′his and 3′g7.

The term “protein” interchangeably used with the term “polypeptide” as used herein describes a group of molecules consisting of more than 30 amino acids, whereas the term “peptide” describes molecules consisting of up to 30 amino acids. Proteins and peptides may further form dimers, trimers and higher oligomers, i.e. consisting of more than one (poly)peptide molecule. Protein or peptide molecules forming such dimers, trimers etc. may be identical or non-identical. The corresponding higher order structures are, consequently, termed homo- or heterodimers, homo- or heterotrimers etc. The terms “protein” and “peptide” also refer to naturally modified proteins or peptides wherein the modification is effected e.g. by glycosylation, acetylation, phosphorylation and the like. Such modifications are well known in the art.

The term “heterologous” refers to the relationship between two or more nucleic acid or protein sequences that are derived from different sources. For example, a promoter is heterologous with respect to an operably linked DNA region, such as a coding sequence if such a combination is not normally found in nature. In addition, a particular sequence may be “heterologous” with respect to a cell or organism into which it is inserted (i.e. does not naturally occur in that particular cell or organism).

The term “recombinant gene” refers to any gene that contains: a) DNA sequences, including regulatory and coding sequences that are not found together in nature, or b) sequences encoding parts of proteins not naturally adjoined, or c) parts of promoters that are not naturally adjoined. Accordingly, a recombinant gene may comprise regulatory sequences and coding sequences that are derived from different sources, or comprise regulatory sequences, and coding sequences derived from the same source, but arranged in a manner different from that found in nature.

Any of the promoters and heterologous nucleic acid sequences described above may be provided in a recombinant vector. A recombinant vector typically comprises, in a 5′ to 3′ orientation: a promoter to direct the transcription of a nucleic acid sequence and a nucleic acid sequence. The recombinant vector may further comprise a 3′ transcriptional terminator, a 3′ polyadenylation signal, other untranslated nucleic acid sequences, transit and targeting nucleic acid sequences, selectable markers, enhancers, and operators, as desired. The wording “5′ UTR” refers to the untranslated region of DNA upstream, or 5′ of the coding region of a gene and “3′ UTR” refers to the untranslated region of DNA downstream, or 3′ of the coding region of a gene. Means for preparing recombinant vectors are well known in the art. Methods for making recombinant vectors particularly suited to plant transformation are described in U.S. Pat. Nos. 4,971,908, 4,940,835, 4,769,061 and 4,757,011. Typical vectors useful for expression of nucleic acids in higher plants are well known in the art and include vectors derived from the tumor-inducing (Ti) plasmid of Agrobacterium tumefaciens. One or more additional promoters may also be provided in the recombinant vector. These promoters may be operably linked, for example, without limitation, to any of the nucleic acid sequences described above. Alternatively, the promoters may be operably linked to other nucleic acid sequences, such as those encoding transit peptides, selectable marker proteins, or antisense sequences. These additional promoters may be selected on the basis of the cell type into which the vector will be inserted. Also, promoters which function in bacteria, yeast, and plants are all well taught in the art. The additional promoters may also be selected on the basis of their regulatory features. Examples of such features include enhancement of transcriptional activity, inducibility, tissue specificity, and developmental stage-specificity.

The recombinant vector may also contain one or more additional nucleic acid sequences. These additional nucleic acid sequences may generally be any sequences suitable for use in a recombinant vector. Such nucleic acid sequences include, without limitation, any of the nucleic acid sequences, and modified forms thereof, described above. The additional structural nucleic acid sequences may also be operably linked to any of the above described promoters. The one or more structural nucleic acid sequences may each be operably linked to separate promoters. Alternatively, the structural nucleic acid sequences may be operably linked to a single promoter (i.e. a single operon).

Yet another embodiment provides a host cell, such as an E. coli cell, an Agrobacterium cell, a yeast cell, or a plant cell, comprising the isolated nucleic acid according to the invention, or the recombinant gene according to the invention.

Other nucleic acid sequences may also be introduced into the host cell along with the promoter and structural nucleic acid sequence, e. g. also in connection with the vector of the invention. These other sequences may include 3′ transcriptional terminators, 3′ polyadenylation signals, other untranslated nucleic acid sequences, transit or targeting sequences, selectable markers, enhancers, and operators. Preferred nucleic acid sequences of the present invention, including recombinant vectors, structural nucleic acid sequences, promoters, and other regulatory elements, are described above.

In further embodiments, a plant and a plant cell are provided comprising the recombinant gene according to the invention. Yet a further embodiment provides seeds obtainable from the plant according to the invention. In another embodiment, the plants or seeds according to the invention are seed crop plants or seeds.

The plant cell or plant comprising the recombinant gene according to the invention can be a plant cell or a plant comprising a recombinant gene of which either the promoter, or the heterologous nucleic acid sequence operably linked to said promoter, are heterologous with respect to the plant cell. Such plant cells or plants may be transgenic plant in which the recombinant gene is introduced via transformation. Alternatively, the plant cell or plant may comprise the promoter according to the invention derived from the same species operably linked to a nucleic acid which is also derived from the same species, i.e. neither the promoter nor the operably linked nucleic acid is heterologous with respect to the plant cell, but the promoter is operably linked to a nucleic acid to which it is not linked in nature. A recombinant gene can be introduced in the plant or plant cell via transformation, such that both the promoter and the operably linked nucleotide are at a position in the genome in which they do not occur naturally. Alternatively, the promoter according to the invention can be integrated in a targeted manner in the genome of the plant or plant cell upstream of an endogenous nucleic acid encoding an expression product of interest, i.e. to modulate the expression pattern of an endogenous gene. The promoter that is integrated in a targeted manner upstream of an endogenous nucleic acid can be integrated in cells of a plant species from which it is originally derived, or in cells of a heterologous plant species. Alternatively, a heterologous nucleic acid can be integrated in a targeted manner in the genome of the plant or plant cell downstream of the promoter according to the invention, such that said heterologous nucleic acid is expressed seed-specifically and embryo-preferentially. Said heterologous nucleic acid is a nucleic acid which is heterologous with respect to the promoter, i.e. the combination of the promoter with said heterologous nucleic acid is not normally found in nature. Said heterologous nucleic acid may be a nucleic acid which is heterologous to said plant species in which it is inserted, but it may also naturally occur in said plant species at a different location in the plant genome. Said promoter or said heterologous nucleic acid can be integrated in a targeted manner in the plant genome via targeted sequence insertion, using, for example, the methods as described in WO2005/049842.

Plants comprising at least two recombinant genes according to the invention wherein the nucleic acid comprising seed-specific and embryo-preferential promoter activity is different in each recombinant gene are, for example, plants comprising a first recombinant gene comprising a nucleotide sequence having at least 95% sequence identity to SEQ ID NO: 2 or a functional fragment thereof, and a second recombinant gene comprising a nucleotide sequence having at least 95% sequence identity to any one of SEQ ID NO: 3 to SEQ ID NO: 10 or a functional fragment thereof. It will be clear that, when the first recombinant gene comprises a nucleotide sequence having at least 95% sequence identity to SEQ ID NO: x or a functional fragment thereof, wherein SEQ ID NO: x is selected from any one of SEQ ID NO: 2 to SEQ ID NO: 10, the second recombinant gene may comprise a nucleotide sequence having at least 95% sequence identity to any one of the sequences according to the invention or a functional fragment thereof, except to SEQ ID NO: x. Said plants are suitable to express different genes with the same tissue-specificity, however without the negative features associated with the repeated use of one promoter, such as gene silencing or recombination of a vector comprising the recombinant genes. The at least two recombinant genes according to the invention may be present at one locus in the genome of said plant, and may be derived from the same transforming DNA molecule.

Plants according to the invention may comprise one or more recombinant genes according to the invention, but may in addition contain a recombinant gene comprising a nucleic acid comprising promoter activity which is preferential or specific to other plant tissues, such as apical meristem, flower buds, cotyledons, flowers, pods, roots, and leaves or other seed developmental stages, operably linked to a nucleic acid sequence encoding an expression product of interest. The recombinant gene according to the invention and the recombinant gene comprising a nucleic acid comprising another promoter activity may be present at one locus and may be derived from the same transforming DNA molecule.

Yet another embodiment provides a method of producing a transgenic plant comprising the steps of (a) introducing or providing the recombinant gene according to the invention to a plant cell to create transgenic cells; and (b) regenerating transgenic plants from said transgenic cell.

“Introducing” in connection with the present application relates to the placing of genetic information in a plant cell or plant by artificial means. This can be effected by any method known in the art for introducing RNA or DNA into plant cells, protoplasts, calli, roots, tubers, seeds, stems, leaves, seedlings, embryos, pollen and microspores, other plant tissues, or whole plants. “Introducing” also comprises stably integrating into the plant's genome. Introducing the recombinant gene can be performed by transformation.

The term “transformation” herein refers to the introduction (or transfer) of nucleic acid into a recipient host such as a plant or any plant parts or tissues including plant cells, protoplasts, calli, roots, tubers, seeds, stems, leaves, seedlings, embryos and pollen. Plants containing the transformed nucleic acid sequence are referred to as “transgenic plants”. Transformed, transgenic and recombinant refer to a host organism such as a plant into which a heterologous nucleic acid molecule (e.g. an expression cassette or a recombinant vector) has been introduced. The nucleic acid can be stably integrated into the genome of the plant.

As used herein, the phrase “transgenic plant” refers to a plant having an introduced nucleic acid stably introduced into a genome of the plant, for example, the nuclear or plastid genomes. In other words, plants containing transformed nucleic acid sequence are referred to as “transgenic plants”. Transgenic and recombinant refer to a host organism such as a plant into which a heterologous nucleic acid molecule (e.g. the promoter, the chimeric gene or the vector as described herein) has been introduced. The nucleic acid can be stably integrated into the genome of the plant.

Transformation methods are well known in the art and include Agrobacterium-mediated transformation. Agrobacterium-mediated transformation of cotton has been described e.g. in U.S. Pat. No. 5,004,863, in U.S. Pat. No. 6,483,013 and WO2000/71733. Plants may also be transformed by particle bombardment: Particles of gold or tungsten are coated with DNA and then shot into young plant cells or plant embryos. This method also allows transformation of plant plastids. Viral transformation (transduction) may be used for transient or stable expression of a gene, depending on the nature of the virus genome. The desired genetic material is packaged into a suitable plant virus and the modified virus is allowed to infect the plant. The progeny of the infected plants is virus free and also free of the inserted gene. Suitable methods for viral transformation are described or further detailed e. g. in WO 90/12107, WO 03/052108 or WO 2005/098004. Further suitable methods well-known in the art are microinjection, electroporation of intact cells, polyethyleneglycol-mediated protoplast transformation, electroporation of protoplasts, liposome-mediated transformation, silicon-whiskers mediated transformation etc. Said transgene may be stably integrated into the genome of said plant cell, resulting in a transformed plant cell. The transformed plant cells obtained in this way may then be regenerated into mature fertile transformed plants.

Further provided is a method of effecting late stage seed-specific and embryo-preferential expression of a nucleic acid comprising introducing the recombinant gene according to the invention into the genome of a plant, or providing the plant according to the invention. Also provided is a method for altering seed properties of a plant or to produce a commercially relevant product in a plant, comprising introducing the recombinant gene according to the invention into the genome of a plant, or providing the plant according to the invention. In another embodiment, said plant is a seed crop plant.

“Seed properties” as used herein are properties of the seed. Seed properties can, for example, be seed yield, seed storage compound production, seed compound accumulation, seed nutrient accumulation; seed micronutrient accumulation; seed storage compound quality, seed compound composition, seed quality, biotic stress tolerance such as disease tolerance, abiotic stress tolerance, herbicide tolerance, seed dormancy, seed imbibition, seed germination, seed vigor. Seed storage compounds can, for example, be, seed oil, seed starch, or seed protein.

Seed properties may be modulated by modulating metabolic pathways, such as starch metabolism, sugar metabolism, inositol phosphate metabolism, glycolysis, amino acid biosynthesis, carbon metabolism, nucleotide metabolism, oxidative pentose phosphate cycle, fatty acid biosynthesis, protein synthesis, or phytate metabolism, and modulating secondary metabolism pathways. Another example is the methyl recycling metabolic activity impacting chromatin remodeling, phospholipid biosynthesis and cell wall lignification. Such metabolic pathways can be modulated by, for example, overexpressing or down-regulating a gene involved in one or more of the metabolic pathways using the early stage seed-specific and embryo-preferential promoter according to the invention.

Yield as used herein can comprise yield of the plant or plant part which is harvested, such as seed, including seed oil content, seed protein content, seed weight, seed number. Increased yield can be increased yield per plant, and increased yield per surface unit of cultivated land, such as yield per hectare. Yield can be increased by modulating, for example, by increasing seed size or oil content or indirectly by increasing the tolerance to biotic and abiotic stress conditions and decreasing seed abortion.

Quality as used herein can comprise quality of the seed or grain such as beneficial carbohydrate composition or level, beneficial amino acid composition or level, beneficial fatty acid composition or level, nutritional value, seed and fiber content.

Abiotic stress tolerance as used herein can comprise resistance to environmental stress factors such as drought, extreme (high or low) temperatures.

Biotic stress tolerance as used herein can comprise pest resistance, such as resistance or fungal, bacterial, bacterial or viral pathogens or insects.

Also provided is the use of the isolated nucleic acid according to the invention to regulate expression of an operably linked nucleic acid in a plant, and the use of the isolated nucleic acid according to the invention, or the recombinant gene according to the invention to alter seed properties of a plant or to produce a commercially relevant product in a plant. In a further embodiment, said plant is a trait as used herein refers to beneficial properties of the plant, such as commercially beneficial properties of a plant.

Also provided is the use of the isolated nucleic acid according to the invention to identify other nucleic acids comprising late stage seed-specific and embryo preferential promoter activity.

The promoters according to the invention can further be used to create hybrid promoters, i.e. promoters containing (parts of) one or more of the promoters(s) of the current invention and (parts of) other promoter which can be newly identified or known in the art. Such hybrid promoters may have optimized tissue specificity or expression level.

Yet another embodiment provides a method of producing food, feed, or an industrial product comprising (a) obtaining the plant or a part thereof, according to the invention; and (b) preparing the food, feed or industrial product from the plant or part thereof. In another embodiment, said food or feed is oil, meal, grain, starch, flour or protein, or said industrial product is biofuel, fiber, industrial chemicals, a pharmaceutical or a nutraceutical.

A “seed crop” or “seed crop plant” as used herein is a crop grown for its seeds or material derived from the seeds. Examples of seed crops are rice, maize, wheat, barley, millet, rye, oats, camelina, crambe, Linum, castor bean, calendula, safflower, sunflower, soybean, cotton, or Brassica species, such as Brassica napus, Brassica juncea, Brassica carinata, Brassica rapa, Brassica oleracea, and Brassica nigra.

“Brassicaceae” or “Brassicaceae plant” as used herein refers to plants belonging to the family of Brassicaceae plants, also called Cruciferae or mustard family. Examples of Brassicaceae are, but are not limited to, Brassica species, such as Brassica napus, Brassica oleracea, Brassica rapa, Brassica carinata, Brassica nigra, and Brassica juncea; Raphanus species, such as Raphanus caudatus, Raphanus raphanistrum, and Raphanus sativus; Matthiola species; Cheiranthus species; Camelina species, such as Camelina sativa; Crambe species, such as Crambe abyssinica and Crambe hispanica; Eruca species, such as Eruca vesicaria; Sinapis species such as Sinapis alba; Diplotaxis species; Lepidium species; Nasturtium species; Orychophragmus species; Armoracia species, Eutrema species; Lepidium species; and Arabidopsis species.

Said Brassicaceae plant can be a Brassica plant. “Brassica plant” refers to allotetraploid or amphidiploid Brassica napus (MCC, 2n=38), Brassica juncea (AABB, 2n=36), Brassica carinata (BBCC, 2n=34), or to diploid Brassica rapa (syn. B. campestris) (AA, 2n=20), Brassica oleracea (CC, 2n=18) or Brassica nigra (BB, 2n=16).

Crop plants of the Brassica species are, for example, Brassica napus, Brassica juncea, Brassica carinata, Brassica rapa (syn. B. campestris), Brassica oleracea or Brassica nigra.

The plants according to the invention may additionally contain an endogenous or a transgene, which confers herbicide resistance, such as the bar or pat gene, which confer resistance to glufosinate ammonium (Liberty®, Basta® or Ignite®) [EP 0 242 236 and EP 0 242 246 incorporated by reference]; or any modified EPSPS gene, such as the 2mEPSPS gene from maize [EPO 508 909 and EP 0 507 698 incorporated by reference], or glyphosate acetyltransferase, or glyphosate oxidoreductase, which confer resistance to glyphosate (RoundupReady®), or bromoxynitril nitrilase to confer bromoxynitril tolerance, or any modified AHAS gene, which confers tolerance to sulfonylureas, imidazolinones, sulfonylaminocarbonyltriazolinones, triazolopyrimidines or pyrimidyl(oxy/thio)benzoates, such as oilseed rape imidazolinone-tolerant mutants PM1 and PM2, currently marketed as Clearfield® canola. Further, the plants according to the invention may additionally contain an endogenous or a transgene which confers increased oil content or improved oil composition, such as a 12:0 ACP thioesteraseincrease to obtain high laureate, which confers pollination control, such as such as barnase under control of an anther-specific promoter to obtain male sterility, or barstar under control of an anther-specific promoter to confer restoration of male sterility, or such as the Ogura cytoplasmic male sterility and nuclear restorer of fertility.

The plants or seeds of the plants according to the invention may be further treated with a chemical compound, such as a chemical compound selected from the following lists: Herbicides: Clethodim, Clopyralid, Diclofop, Ethametsulfuron, Fluazifop, Glufosinate, Glyphosate, Metazachlor, Quinmerac, Quizalofop, Tepraloxydim, Trifluralin. Fungicides/PGRs: Azoxystrobin, N-[9-(dichloromethylene)-1,2,3,4-tetrahydro-1,4-methanonaphthalen-5-yl]-3-(difluoromethyl)-1-methyl-1H-pyrazole-4-carboxamide (Benzovindiflupyr, Benzodiflupyr), Bixafen, Boscalid, Carbendazim, Carboxin, Chlormequat-chloride, Coniothryrium minitans, Cyproconazole, Cyprodinil, Difenoconazole, Dimethomorph, Dimoxystrobin, Epoxiconazole, Famoxadone, Fluazinam, Fludioxonil, Fluopicolide, Fluopyram, Fluoxastrobin, Fluquinconazole, Flusilazole, Fluthianil, Flutriafol, Fluxapyroxad, Iprodione, Isopyrazam, Mefenoxam, Mepiquat-chloride, Metalaxyl, Metconazole, Metominostrobin, Paclobutrazole, Penflufen, Penthiopyrad, Picoxystrobin, Prochloraz, Prothioconazole, Pyraclostrobin, Sedaxane, Tebuconazole, Tetraconazole, Thiophanate-methyl, Thiram, Triadimenol, Trifloxystrobin, Bacillus firmus, Bacillus firmus strain I-1582, Bacillus subtilis, Bacillus subtilis strain GB03, Bacillus subtilis strain QST 713, Bacillus pumulis, Bacillus. pumulis strain GB34. Insecticides: Acetamiprid, Aldicarb, Azadirachtin, Carbofuran, Chlorantraniliprole (Rynaxypyr), Clothianidin, Cyantraniliprole (Cyazypyr), (beta-)Cyfluthrin, gamma-Cyhalothrin, lambda-Cyhalothrin, Cypermethrin, Deltamethrin, Dimethoate, Dinetofuran, Ethiprole, Flonicamid, Flubendiamide, Fluensulfone, Fluopyram,Flupyradifurone, tau-Fluvalinate, Imicyafos, Imidacloprid, Metaflumizone, Methiocarb, Pymetrozine, Pyrifluquinazon, Spinetoram, Spinosad, Spirotetramate, Sulfoxaflor, Thiacloprid, Thiamethoxam, 1-(3-chloropyridin-2-yl)-N-[4-cyano-2-methyl-6-(methylcarbamoyl)phenyl]-3-{[5-(trifluoromethyl)-2H-tetrazol-2-yl]methyl}-1H-pyrazole-5-carboxamide, 1-(3-chloropyridin-2-yl)-N-[4-cyano-2-methyl-6-(methylcarbamoyl)phenyl]-3-{[5-(trifluoromethyl)-1H-tetrazol-1-yl]methyl}-1H-pyrazole-5-carboxamide, 1-{2-fluoro-4-methyl-5-[(2,2,2-trifluorethyl)sulfinyl]phenyl}-3-(trifluoromethyl)-1H-1,2,4-triazol-5-amine, (1E)-N-[(6-chloropyridin-3-yl)methyl]-N′-cyano-N-(2,2-difluoroethyl)ethanimidamide, Bacillus firmus, Bacillus firmus strain 1-1582, Bacillus subtilis, Bacillus subtilis strain GB03, Bacillus subtilis strain QST 713, Metarhizium anisopliae F52.

Whenever reference to a “plant” or “plants” according to the invention is made, it is understood that also plant parts (cells, tissues or organs, seed pods, seeds, severed parts such as roots, leaves, flowers, pollen, etc.), progeny of the plants which retain the distinguishing characteristics of the parents, such as seed obtained by selfing or crossing, e.g. hybrid seed (obtained by crossing two inbred parental lines), hybrid plants and plant parts derived there from are encompassed herein, unless otherwise indicated.

In some embodiments, the plant cells of the invention as well as plant cells generated according to the methods of the invention, may be non-propagating cells.

The obtained plants according to the invention can be used in a conventional breeding scheme to produce more plants with the same characteristics or to introduce the same characteristic in other varieties of the same or related plant species, or in hybrid plants. The obtained plants can further be used for creating propagating material.

Plants according to the invention can further be used to produce gametes, seeds (including crushed seeds and seed cakes), seed oil, embryos, either zygotic or somatic, progeny or hybrids of plants obtained by methods of the invention. Seeds obtained from the plants according to the invention are also encompassed by the invention.

“Creating propagating material”, as used herein, relates to any means know in the art to produce further plants, plant parts or seeds and includes inter alia vegetative reproduction methods (e.g. air or ground layering, division, (bud) grafting, micropropagation, stolons or runners, storage organs such as bulbs, corms, tubers and rhizomes, striking or cutting, twin-scaling), sexual reproduction (crossing with another plant) and asexual reproduction (e.g. apomixis, somatic hybridization).

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a nucleic acid which is functionally or structurally defined, may comprise additional DNA regions etc.

Furthermore, the disclosed invention is expected to yield similar results in other seed crop plant species. Particularly, it is expected to drive late stage seed-specific and embryo-preferential expression in soybean. It is also expected to drive late stage seed-specific and embryo-preferential expression in wheat. The disclosed promoter may lead to a late stage seed-specific and embryo-preferential expression in cotton.

The sequence listing contained in the file named “BCS16-2004_ST25.txt”, which is 84 kilobytes (size as measured in Microsoft Windows®), contains 41 sequences SEQ ID NO: 1 through SEQ ID NO: 41 is filed herewith by electronic submission and is incorporated by reference herein.

In the description and examples, reference is made to the following sequences:

### SEQUENCES

SEQ ID NO: 1: nucleotide sequence of the T-DNA PcruP2 BnA2::GUS.

SEQ ID NO: 2: nucleotide sequence of the promoter PcruP2-4 BnA2.

SEQ ID NO: 3: nucleotide sequence of the promoter PcruP2-4 BnA1.

SEQ ID NO: 4: nucleotide sequence of the promoter PcruP2-4 BnC1.

SEQ ID NO: 5: nucleotide sequence of the promoter PcruP2-4 Br2.

SEQ ID NO: 6: nucleotide sequence of the promoter PcruP2-4 Br1.

SEQ ID NO: 7: nucleotide sequence of the promoter PcruP2-4 Bo.

SEQ ID NO: 8: nucleotide sequence of the promoter PcruP2-4 BjA2.

SEQ ID NO: 9: nucleotide sequence of the promoter PcruP2-4 BjA1.

SEQ ID NO: 10: nucleotide sequence of the promoter PcruP2-4 BjB1.

SEQ ID NO: 11: amino acid sequence of CRUP2 BnA2.

SEQ ID NO: 12: amino acid sequence of CRUP2 BnA1.

SEQ ID NO: 13: amino acid sequence of CRUP2 BnC1.

SEQ ID NO: 14: amino acid sequence of CRUP2 Br2.

SEQ ID NO: 15: amino acid sequence of CRUP2 Br1.

SEQ ID NO: 16: amino acid sequence of CRUP2 Bo.

SEQ ID NO: 17: amino acid sequence of CRUP2 BjA2.

SEQ ID NO: 18: amino acid sequence of CRUP2 BjA1.

SEQ ID NO: 19: amino acid sequence of CRUP2 BjB1.

SEQ ID NO: 20: nucleotide sequence of the coding sequence of CRUP2 BnA2.

SEQ ID NO: 21: nucleotide sequence of the coding sequence of CRUP2 BnA1.

SEQ ID NO: 22: nucleotide sequence of the coding sequence of CRUP2 BnC1.

SEQ ID NO: 23: nucleotide sequence of the coding sequence of CRUP2 Br2.

SEQ ID NO: 24: nucleotide sequence of the coding sequence of CRUP2 Br1.

SEQ ID NO: 25: nucleotide sequence of the coding sequence of CRUP2 Bo.

SEQ ID NO: 26: nucleotide sequence of the coding sequence of CRUP2 BjA2.

SEQ ID NO: 27: nucleotide sequence of the coding sequence of CRUP2 BjA1.

SEQ ID NO: 28: nucleotide sequence of the coding sequence of CRUP2 BjB1.

SEQ ID NO: 29: motif 1.

SEQ ID NO: 30: motif 3.

SEQ ID NO: 31: motif 6.

SEQ ID NO: 32: motif 7.

SEQ ID NO: 33: motif 8.

SEQ ID NO: 34: motif 9.

SEQ ID NO: 35: motif 10.

SEQ ID NO: 36: motif 11.

SEQ ID NO: 37: motif 12.

SEQ ID NO: 38: motif 13.

SEQ ID NO: 39: motif 14.

SEQ ID NO: 40: motif 15.

SEQ ID NO: 41: motif 16.

### EXAMPLES

Unless stated otherwise in the Examples, all recombinant DNA techniques are carried out according to standard protocols as described in Sambrook and Russell (2001) Molecular Cloning: A Laboratory Manual, Third Edition, Cold Spring Harbor Laboratory Press, NY, in Volumes 1 and 2 of Ausubel et al. (1994) Current Protocols in Molecular Biology, Current Protocols, USA and in Volumes I and II of Brown (1998) Molecular Biology LabFax, Second Edition, Academic Press (UK). Standard materials and methods for plant molecular work are described in Plant Molecular Biology Labfax (1993) by R. D. D. Croy, jointly published by BIOS Scientific Publications Ltd (UK) and Blackwell Scientific Publications, UK. Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1—Generation of Expression Constructs with the PcruP2 BnA2 Promoter of Brassica napus Operably Linked to the GUS Reporter Gene (PcruP2 BnA2::GUS)

The promoter sequence of the Brassica napus CRUP2 A2 promoter (SEQ ID NO: 2 or 5′ to 3′ position 139 to 1681 of SEQ ID NO:1) isolated from an in house developed Brassica napus line, the GUS gene (6-glucuronidase) with intron (5′ to 3′ position 1682 to 3682 of SEQ ID NO: 1) and a fragment of the 3′ untranslated region (UTR) of the gene 7 of Agrobacterium tumefaciens octopine Ti plasmid (5′ to 3′ position 3739 to 3942 of SEQ ID NO: 1) were assembled in a vector which contains the bar selectable marker cassette (position 4023 to 6533 of SEQ ID NO: 1) to result in the T-DNA PcruP2 BnA2::GUS (SEQ ID NO: 1).

### Example 2—Generation of Transgenic Plants Comprising the PcruP2 BnA2::GUS

In a next step the recombinant vectors comprising the expression cassette of example 1, i. e. PcruP2 BnA2, were used to stably transform Brassica napus.

### Example 3—in Planta Expression Pattern of PcruP2 BnA2::GUS in Brassica napus

The in planta expression pattern of PcruP2 BnA2::GUS in the different seed tissues and non-seed tissues of Brassica napus seeds was monitored according to the method of Jasik et al. 2011.

No GUS activity was detected in the assessed non-seed tissues, namely young leaves, young stems, flower buds, flowers and pods, thereby confirming the seed-specificity of the selected promoters.

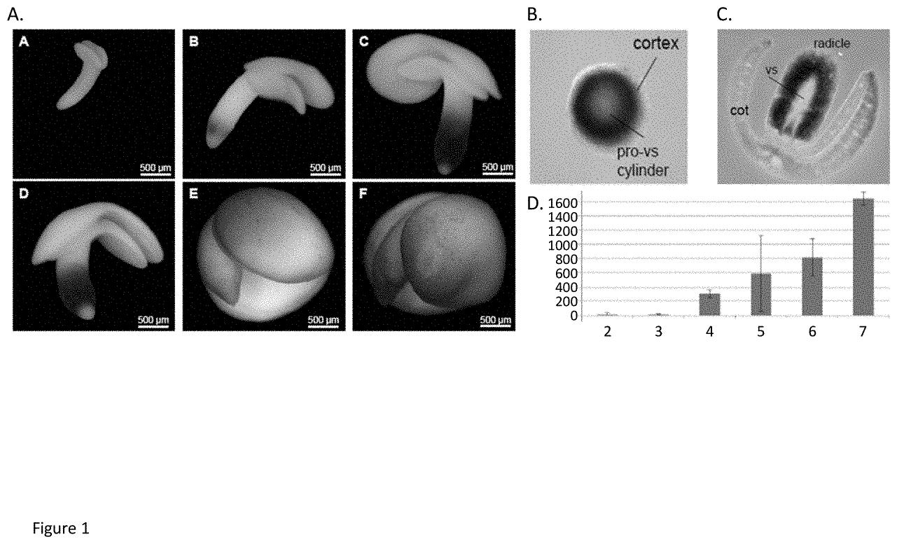

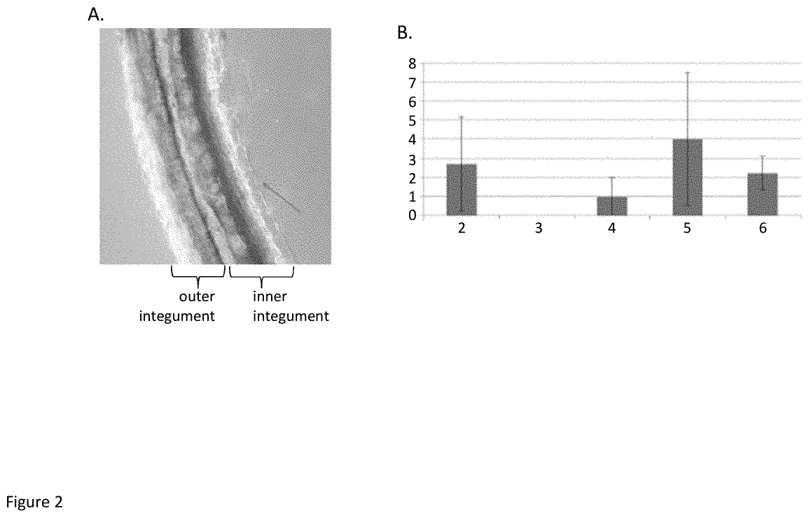

FIG. 1 shows the GUS labelling of the reporter gene in the embryo at different developmental stages. Staining is first detected in the embryos at the “walking stick” cotyledon stage (panel A, sub-panel B). The expression of the reporter gene (GUS) is then restricted to the radicle until the end of the curled cotyledon stage (panel A, sub-panel D). The radicle cap is then not stained. Transverse section of the radicle at the curled cotyledon stage indicates that the cortex is intensely stained while the pro-vascular cylinder is less stained (panel B). From the green cotyledon stage on (panel A, sub-panels E and F) the expression of the reporter gene is detected in the radicle, radicle cap included, as well as in the cotyledons. Longitudinal section of a mature embryo (panel C) shows that the vascular tissues of the radicle are not stained. Concerning the staining in the cotyledons, the outer cotyledon shows more intense staining than the inner cotyledon and the parenchyma tissues are preferentially stained over the vascular structures. The semi quantitative assessment of the GUS labelling in the embryo of transgenic lines carrying the PcruP2 BnA2::GUS T-DNA (panel D) shows that the intensity of the embryo staining increases many fold towards maturation.

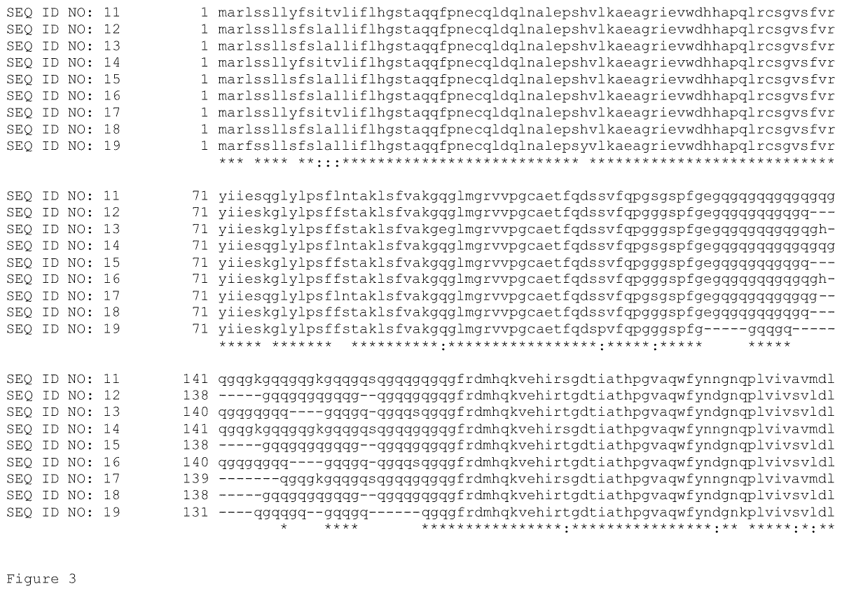

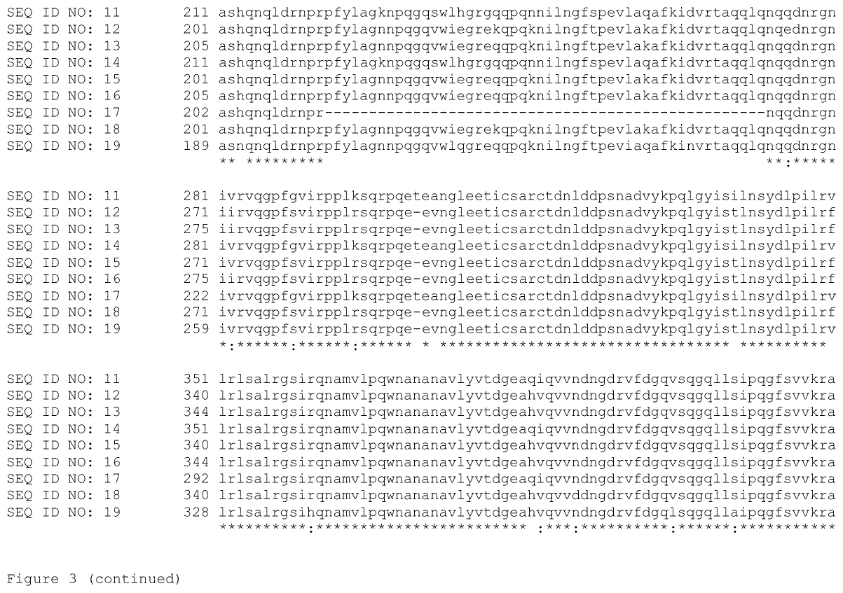

FIG. 2 panel A shows the GUS labelling of the reporter gene in the seed coat at late stage. The seed coat is labelled in the inner integument only. The endosperm is also weakly labelled. Panel B provides the semi quantitative assessment of the GUS labelling in the seed coat of the transgenic lines carrying the PcruP2 BnA2::GUS T-DNA. The GUS staining in the seed coat can be detected at very low level.

The strong staining in the late stage embryo and the weak staining in the seed coat and endosperm indicate that the PcruP2 BnA2 promoter has embryo-preferential promoter activity.

### Example 4—Identification of the Different Brassica napus Copies of CRUP2 BnA2 and of the Orthologues of CRUP2 in Brassica rapa, Brassica Oleracea and Brassica juncea

The sequences of the different Brassica napus copies of CRUP2 as well as their orthologues in Brassica rapa, Brassica oleracea and Brassica juncea were obtained by blasting the coding sequence of the CRUP2 BnA2 against an in-house database of Brassica napus, Brassica rapa, Brassica oleracea and Brassica juncea sequences.

The nucleotide sequences obtained in this way are given in SEQ ID NO: 20 to SEQ ID NO: 28. These nucleotide sequences were translated into amino acid sequences, given in SEQ ID NO: 11 to SEQ ID NO: 19.

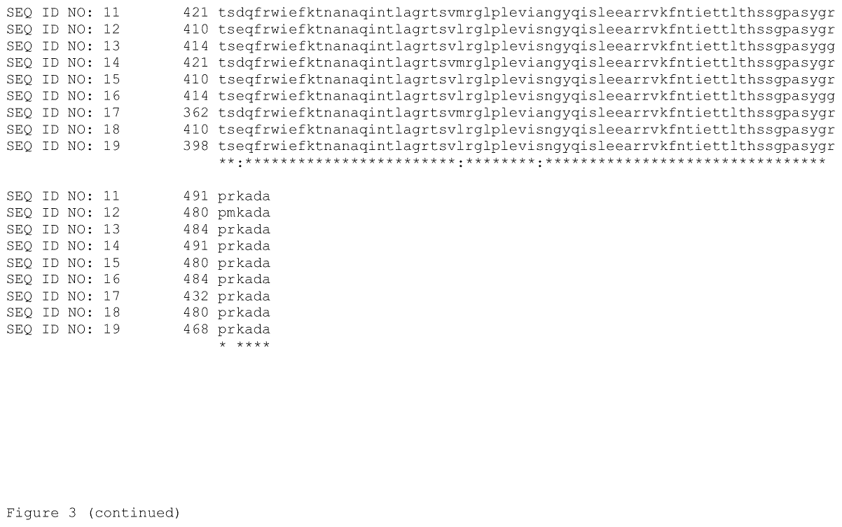

FIG. 3 shows the alignment of the retrieved amino acid sequence. Any two of these sequences share at least 83% identity.

### Example 5—RNA Isolation from Different Tissues of Brassica napus and Brassica juncea

The following tissues were isolated from Brassica napus:
a. Apical meristem 33 days after sowing (DAS) (including smallest leaves) (AM33)
b. Big flower buds (>5 mm) 42 DAS (BFB42)
c. Cotyledons (with hypocotyl) 10 DAS (CTYL10)
d. Open flowers 52 DAS (OF52)
e. Pods 14-20 DAS (Pod2)
f. Pods 21-25 DAS (Pod3)
g. Roots 14 DAS (Ro2w)
h. Small flower buds 5 mm 42 DAS (SFB42)
i. Seeds 14-20 days after flowering (DAF) (Seed2)
j. Seeds 21-25 DAF (Seed3)
k. Seeds 26-30 DAF (Seed4)
l. Seeds 31-35 DAF (Seed5)
m. Seeds 42 DAF (Seed6)
n. Seeds 49 DAF (Seed7)
o. Stem 14 DAS (St2w)
p. Stem 33 DAS (St5w)
q. Young leaf 33 DAS 3 cm leaf next to apical meristem) (YL33)

The following tissues were isolated from Brassica juncea:
a. Apical meristem 22 days after sowing (DAS) (including smallest leaves) (AM22)
b. Big flower buds (>5 mm) 35 DAS (BFB35)
c. Cotyledons (with hypocotyl) 8 DAS (CTYL8)
d. Open flowers 35 DAS (OF35)
e. Pods 14-20 DAS (Pod2)
f. Pods 21-25 DAS (Pod3)
g. Pods 26-30 DAS (Pod4)
h. Pods 31-35 DAS (Pod5)
i. Roots 14 DAS (Ro2w)
j. Small flower buds 5 mm 35 DAS (SFB35)
k. Seeds 14-20 days after flowering (DAF) (Seed2)
l. Seeds 21-25 DAF (Seed3)
m. Seeds 26-30 DAF (Seed4)
n. Seeds 31-35 DAF (Seed5)
o. Seeds 42 DAF (Seed6)
p. Seeds 49 DAF (Seed7)
q. Stem 14 DAS (St2w)
r. Stem 22 DAS (St3w)
s. Young leaf 22 DAS 3 cm leaf next to apical meristem) (YL22)
t. Old leaf 22 DAS (OL22)

The following seed sub-tissues were isolated from Brassica napus:
a. Endosperm, 18 days after flowering (DAF)
b. Endosperm, 24 DAF
c. Embryonic hypocotyl, 18 DAF
d. Embryonic hypocotyl, 24 DAF
e. Embryonic hypocotyl, 28 DAF
f. Embryonic hypocotyl, 32 DAF
g. Embryonic hypocotyl, 46 DAF
h. Embryonic inner cotyledon, 18 DAF
i. Embryonic inner cotyledon, 24 DAF
j. Embryonic inner cotyledon, 28 DAF
k. Embryonic inner cotyledon, 32 DAF
I. Embryonic inner cotyledon, 46 DAF
m. Embryonic outer cotyledon, 18 DAF
n. Embryonic outer cotyledon(inner part), 24 DAF
o. Embryonic outer cotyledon(inner part), 28 DAF
p. Embryonic outer cotyledon(inner part), 32 DAF
q. Embryonic outer cotyledon(inner part), 46 DAF
r. Embryonic outer cotyledon(outer part), 24 DAF
s. Embryonic outer cotyledon(outer part), 28 DAF
t. Embryonic outer cotyledon(outer part), 32 DAF
u. Embryonic outer cotyledon(outer part), 46 DAF

For the isolation of the seed sub-tissues, freshly harvested seeds were frozen at −80° C. and cut into 20 μm sections. Sections were placed on PET-membranes, lyophilized at −20° C., and then used for laser-assisted microdissection (PALM Laser-Microbeam instrument; Bernried/Germany) (for details see Schiebold et al., 2011, Plant Methods 7:19). Up to 5 distinct embryonic tissues plus endosperm were targeted. Tissue dissection was applied to seeds at 18, 24, 28, 32 and 46 DAF, covering the developmental period from onset of storage activity until late maturation. RNA was extracted (purification of total RNA by RNeasy Micro kit; Qiagen) and amplified (C&E version ExpressArt mRNA amplification Nano kit; Amp-tec) as detailed in Schiebold et al. 2011 (supra).

Total RNA from the non-seed sub-tissues was isolated according to standard methods.

In our growth conditions, the correspondence between embryo developmental stages and the selected time points is as follows:
a. Between 10 and 13 DAF: torpedo stage
b. Seed2 or between 14 and 20 DAF: “walking stick” cotyledon stage
c. Seed3 or between 21 and 25 DAF: curled cotyledon stage
d. Seed4 and Seed5 or between 26 and 35 DAF: green cotyledon stage
e. Seed6 and Seed7 or after 36 DAF: mature embryo

### Example 6—in Silico Expression Analyses of the Different Copies of CRUP2 of Brassica napus and their Orthologues

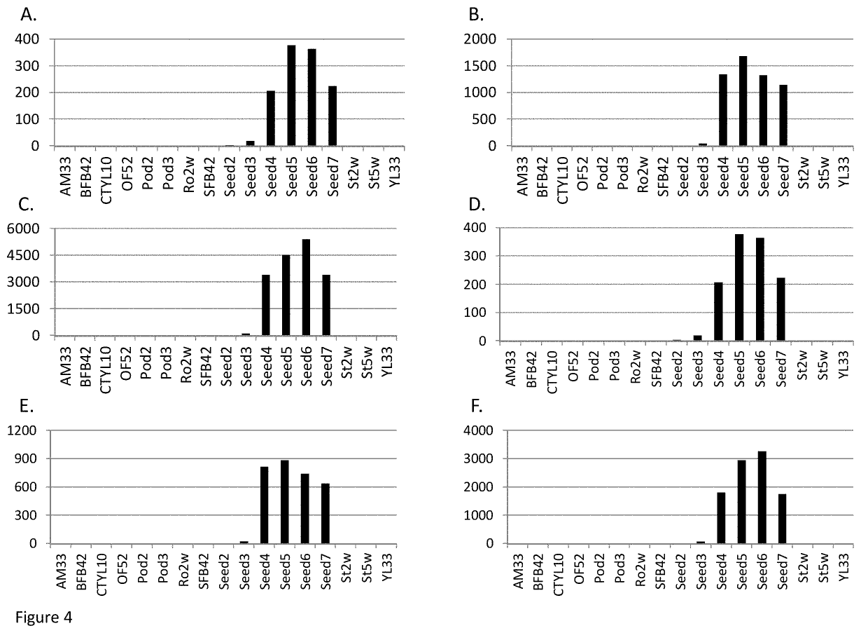

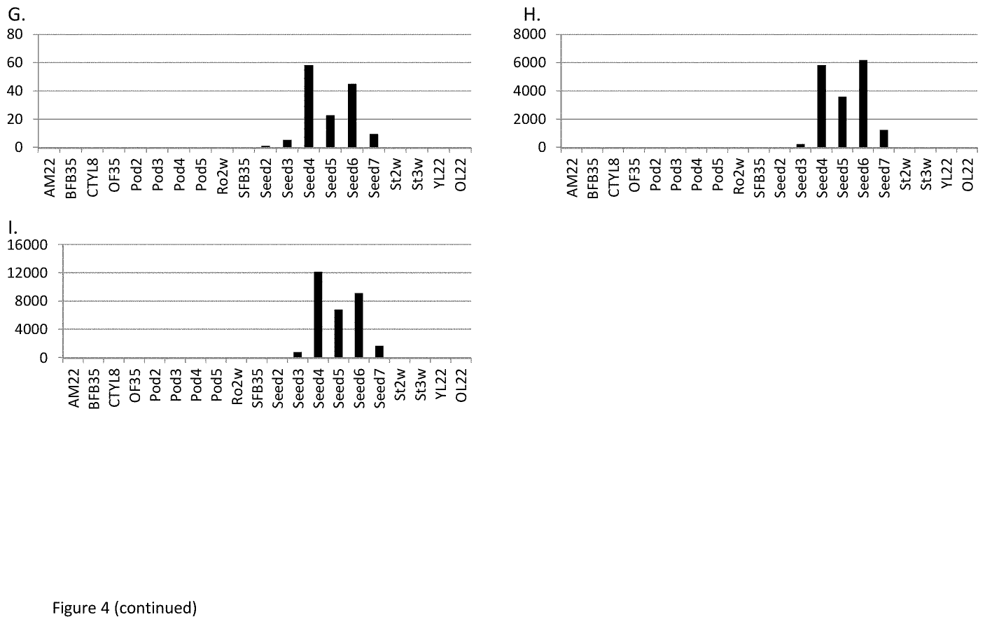

FIG. 4 shows the relative expression levels of the endogenous transcripts of the different Brassica napus (A-C), Brassica rapa (D-E) and Brassica oleracea (F) and Brassica juncea (G-I) copies of CRUP2 in different tissues, as isolated in Example 5.

The CRUP2 BnA2 transcript (panel A) is abundantly detected in the Seed4, Seed5, Seed6 and Seed7 tissues, and only barely detectable in the Seed3 tissues. This result confirms, as determined in planta, that PcruP2 BnA2 has late stage seed-specific promoter activity.

The CRUP2 BnA1 transcript (panel B) is abundantly detected in the Seed4, Seed5, Seed6 and Seed7 tissues, and only barely detectable in the Seed3 tissues. This result indicates that PcruP2 BnA1 has late stage seed-specific promoter activity.

The CRUP2 BnC1 transcript (panel C) is abundantly detected in the Seed4, Seed5, Seed6 and Seed7 tissues, and only barely detectable in the Seed3 tissues. This result indicates that PcruP2 BnC1 has late stage seed-specific promoter activity.

The CRUP2 Br2 transcript (panel D) is abundantly detected in the Seed4, Seed5, Seed6 and Seed7 tissues, and only barely detectable in the Seed2, Seed3, Pod2, Pod3 and 5 week-old stem tissues. This result indicates that PcruP2 Br2 has late stage seed-specific promoter activity.

The CRUP2 Br1 transcript (panel E) is abundantly detected in the Seed4, Seed5, Seed6 and Seed7 tissues, and only barely detectable in the Seed3, Pod2, 2 week-old roots, small flower buds and 5 week-old stem tissues. This result indicates that PcruP2 Br1 has late stage seed-specific promoter activity.

The CRUP2 Bo transcript (panel F) is abundantly detected in the Seed4, Seed5, Seed6 and Seed7 tissues, and only barely detectable in the Seed2, Seed3, Pod2, Pod3, cotyledons, 2 week-old roots, 5 week-old leaf and small flower buds tissues. This result indicates that PcruP2 Bo has late stage seed-specific promoter activity.

The CRUP2 BjA2 transcript (panel G) is clearly detected in the Seed4, Seed5 and Seed6 tissues, mildly detected in the Seed7 tissues and only barely detectable in the Seed2, Seed3 and Pod2 tissues. This result indicates that PcruP2 BjA2 has late stage seed-specific promoter activity.

The CRUP2 BjA1 transcript (panel H) is abundantly detected in the Seed4, Seed5, Seed6 and Seed7 tissues, and only barely detectable in the Seed2, Seed3, Pod2, Pod3, Pod4, Pod5, apical meristem, cotyledons, open flowers and old leaves tissues. This result indicates that PcruP2 BjA1 has late stage seed-specific promoter activity.

The CRUP2 BjB1 transcript is abundantly detected in the Seed4, Seed5 and Seed6 tissues, mildly detected in the Seed3 and Seed7 tissues and only barely detectable in the Seed2, Pod2, Pod3, Pod4, Pod5, apical meristem, cotyledons, open flowers and old leaves tissues. This result indicates that PcruP2 BjB1 has late stage seed-specific promoter activity.

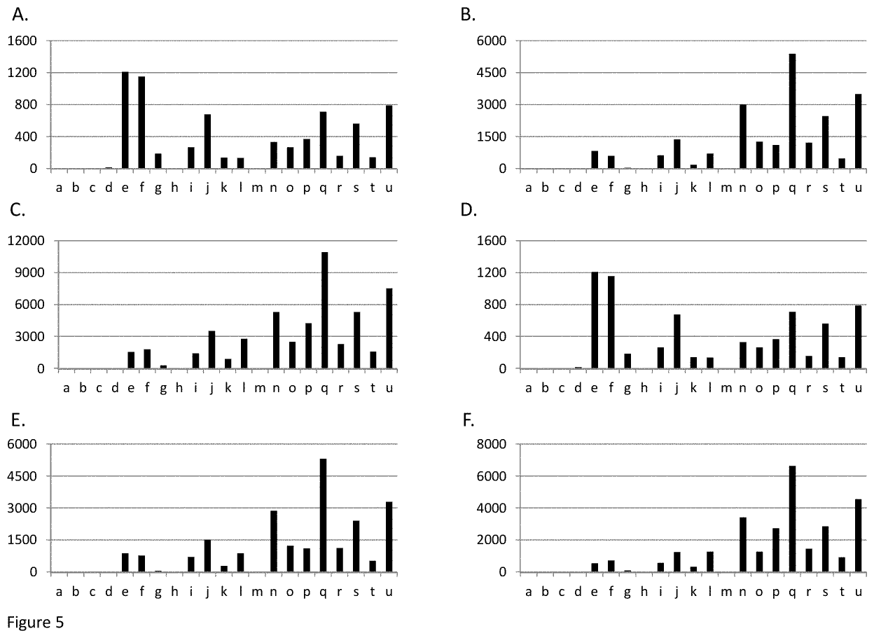

FIG. 5 shows the relative expression levels of the endogenous transcripts of the different Brassica napus (A-C), Brassica rapa (D-E) and Brassica oleracea (F) copies of CRUP2 in different seed sub-tissues, as isolated in Example 5.

The CRUP2 BnA2 transcript (panel A) is abundantly detected in the embryo tissues (except at 18 DAF), but is absent from the endosperm tissues. This result confirms, as determined in planta, that PcruP2 BnA2 has embryo-preferential promoter activity.

The CRUP2 BnA1 transcript (panel B) is abundantly detected in the embryo tissues (except at 18 DAF), but is absent from the endosperm tissues. This result indicates that PcruP2 BnA1 has embryo-preferential promoter activity.

The CRUP2 BnC1 transcript (panel C) is abundantly detected in the embryo tissues (except at 18 DAF), but is absent from the endosperm tissues. This result indicates that PcruP2 BnC1 has embryo-preferential promoter activity.

The CRUP2 Br2 transcript (panel D) is abundantly detected in the embryo tissues (except at 18 DAF), but is absent from the endosperm tissues. This result indicates that PcruP2 Br2 has embryo-preferential promoter activity.

The CRUP2 Br1 transcript (panel E) is abundantly detected in the embryo tissues (except at 18 DAF), but is absent from the endosperm tissues. This result indicates that PcruP2 Br1 has embryo-preferential promoter activity.

The CRUP2 Bo transcript (panel F) is abundantly detected in the embryo tissues (except at 18 DAF), but is absent from the endosperm tissues. This result indicates that PcruP2 Bo has embryo-preferential promoter activity.

### Example 7—Sequence Analysis of the Promoters of the CRUP2 Genes from Brassica rapa, Brassica Juncea, Brassica oleracea and Brassica napus

For each CRUP2 gene identified the about 1.5 kb of genomic DNA sequence upstream of the translation start was retrieved from an in-house database of Brassica napus, Brassica rapa, Brassica oleracea and Brassica juncea sequences. The nucleotide sequences obtained in this way are given in SEQ ID NO: 3 to SEQ ID NO: 10.

A promoter analysis was carried out using publicly available databases such as PLACE (www.dna.affrc.go.jp/PLACE/), RegSite (linux1.softberry.com/berry.phtml?topic=regsitelist), PlantCare (Lescot et al., 2002; available at bioinformatics.psb.ugent.be/webtools/plantcare/html/) and AtcisDB (Davuluri et al., 2003). The search was limited to seed-specific elements and two RY-repeat elements could be predicted in all promoters disclosed herein. The exact sequence of the RY-repeat element is CATGCA and was observed from position 1310 and from position 1327 on SEQ ID NO: 2, from position 1215 and from position 1232 on SEQ ID NO: 3, 4, 6, 9 and 10, from position 1267 and from position 1284 on SEQ ID NO: 5, from position 1216 and from position 1233 on SEQ ID NO: 7, from position 1214 and from position 1231 on SEQ ID NO: 8.

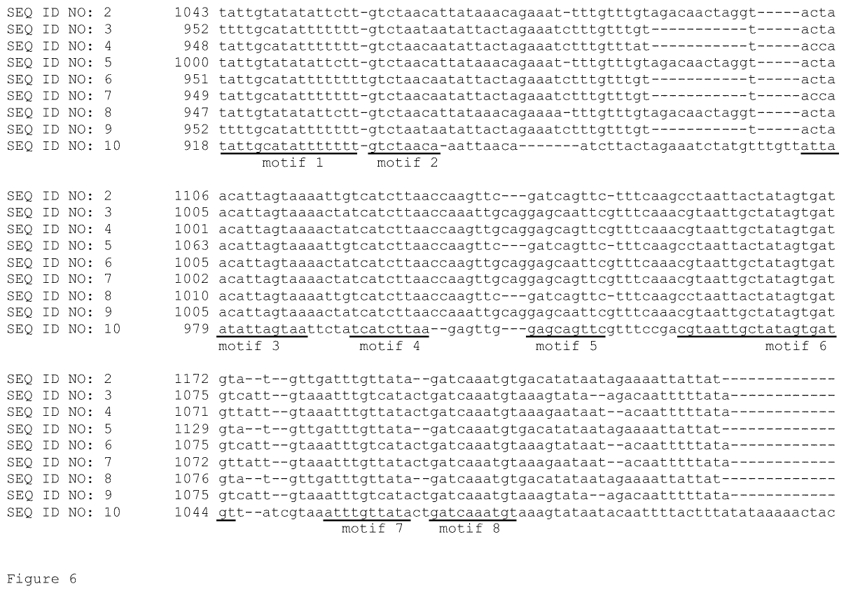

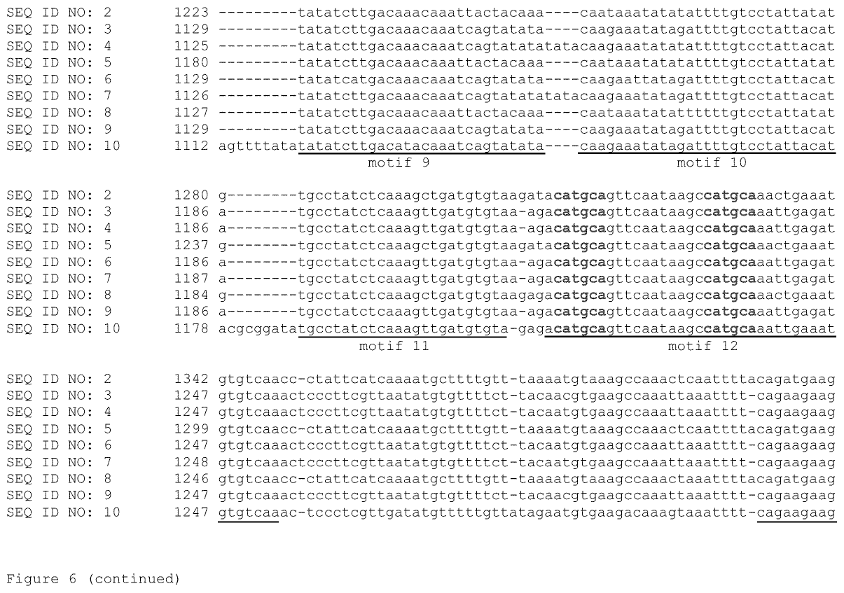

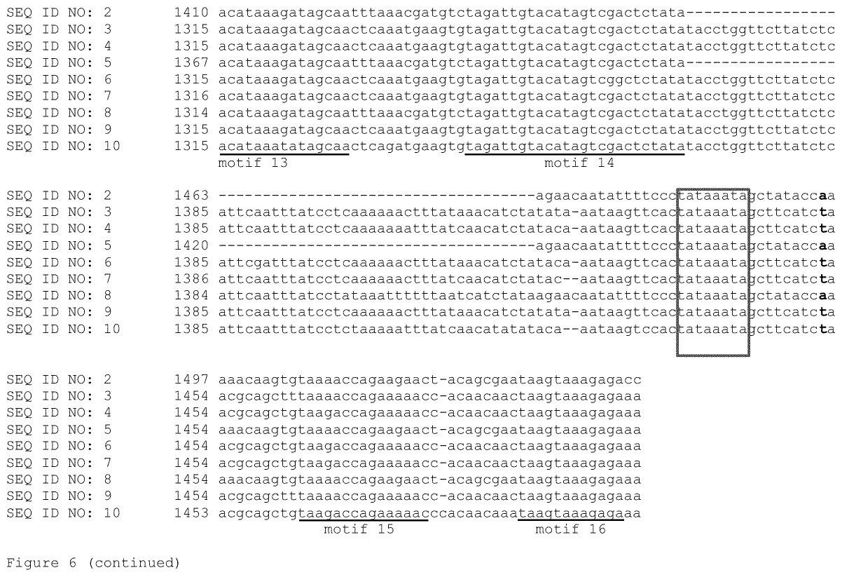

FIG. 6 shows the alignment of the 3′ end sequence of the promoter sequences (SEQ ID NO: 2 to SEQ ID NO: 10). These promoters share a surprisingly high level of conservation in this region. Sixteen consensus sequences (motifs) were identified. The promoters comprise the following motifs in their 3′ about 500 bp sequence:
a. motif 1 is given in SEQ ID NO: 29;
b. motif 2 has the sequence gtctaaya;
c. motif 3 is given in SEQ ID NO: 30;
d. motif 4 has the sequence tcatcttaa;
e. motif 5 has the sequence gakcarttc;
f. motif 6 is given in SEQ ID NO: 31;
g. motif 7 is given in SEQ ID NO: 32;
h. motif 8 is given in SEQ ID NO: 33;
i. motif 9 is given in SEQ ID NO: 34;
j. motif 10 is given in SEQ ID NO: 35;
k. motif 11 is given in SEQ ID NO: 36;
l. motif 12 is given in SEQ ID NO: 37;
m. motif 13 is given in SEQ ID NO: 38;
n. motif 14 is given in SEQ ID NO: 39;
o. motif 15 is given in SEQ ID NO: 40;
p. motif 16 is given in SEQ ID NO: 41.

The high degree of conservation of these motifs in all analyzed promoter sequences described herein indicate that these motifs may be required for the observed seed-specific and embryo-preferential expression pattern.

Consequently, as PcruP2 BjA1 sequence comprises the motifs 1 to 16, it can be concluded that it has embryo-preferential promoter activity. As PcruP2 BjA2 sequence comprises the motifs 1 to 16, it can be concluded that it has embryo-preferential promoter activity. As PcruP2 BjB1 sequence comprises the motifs 1 to 16, it can be concluded that it has embryo-preferential promoter activity.

More generally, these results indicate that a Brassica promoter comprising the motifs 1 to 16 would have late stage seed-specific and embryo-preferential promoter activity.

1. (canceled)

2. (canceled)

3. A recombinant gene comprising a nucleic acid having late stage seed-specific and embryo-preferential promoter activity comprising:
a. a nucleic acid comprising a nucleotide sequence of any one of SEQ ID NO: 2 to SEQ ID NO: 10 or a functional fragment thereof; or
b. a nucleic acid comprising a nucleotide sequence having at least 80% sequence identity to any one of SEQ ID NO: 2 to SEQ ID NO: 10, or a functional fragment thereof, operably linked to a heterologous nucleic acid sequence encoding an expression product of interest, and optionally a transcription termination and polyadenylation sequence.

4. The recombinant gene according to claim 3, wherein the expression product of interest is an RNA molecule capable of modulating the expression of a gene or is a protein.

5. A host cell comprising the recombinant gene according to claim 3.

6. The host cell of claim 5, which is an E. coli cell, an Agrobacterium cell, yeast cell, an algal cell, or a plant cell.

7. A plant comprising the recombinant gene of claim 3.

8. A plant comprising at least two recombinant genes according to claim 3, wherein said nucleic acid having late stage seed-specific and embryo-preferential promoter activity and, optionally, the heterologous nucleic acid sequence, is different in each recombinant gene.

9. Seeds obtainable from the plant according to claim 7.

10. (canceled)

11. A method of producing a transgenic plant comprising:
a. introducing or providing the recombinant gene according to claim 3 to a plant cell to create transgenic cells; and
b. regenerating transgenic plants from said transgenic cell.

12. A method of effecting late stage seed-specific and embryo-preferential expression of a nucleic acid comprising introducing the recombinant gene according to claim 3 into the genome of a plant.

13. A method for altering seed properties of a plant or to produce a commercially relevant product in a plant, comprising introducing the recombinant gene according to claim 3 into the genome of a plant.

14. (canceled)

15. (canceled)

16. (canceled)

17. The method according to claim 11, wherein said plant is a seed crop plant.

18. A method of producing food, feed, or an industrial product comprising
preparing food, feed or industrial product from the plant of claim 7 or part thereof.

19. The method of claim 18, wherein
a) the food or feed is oil, meal, grain, starch, flour or protein; or
b) the industrial product is biofuel, fiber, industrial chemicals, a pharmaceutical or a nutraceutical.

20. The recombinant gene according to claim 3, wherein said nucleic acid having late stage seed-specific and embryo-preferential promoter activity comprises:
a. the nucleotide sequence of SEQ ID NO: 29;
b. the nucleotide sequence gtctaaya;
c. the nucleotide sequence of SEQ ID NO: 30;
d. the nucleotide sequence tcatcttaa;
e. the nucleotide sequence gakcarttc;
f. the nucleotide sequence of SEQ ID NO: 31;
g. the nucleotide sequence of SEQ ID NO: 32;
h. the nucleotide sequence of SEQ ID NO: 33;
i. the nucleotide sequence of SEQ ID NO: 34;
j. the nucleotide sequence of SEQ ID NO: 35;
k. the nucleotide sequence of SEQ ID NO: 36;
l. the nucleotide sequence of SEQ ID NO: 37;
m. the nucleotide sequence of SEQ ID NO: 38;
n. the nucleotide sequence of SEQ ID NO: 39;
o. the nucleotide sequence of SEQ ID NO: 40;
p. the nucleotide sequence of SEQ ID NO: 41 between the nucleotide positions corresponding to the nucleotide position 1043 and the nucleotide position 1543 of SEQ ID NO: 2.

21. The recombinant gene according to claim 3, operably linked to a transcription termination and polyadenylation region functional in plants.

22. A method of effecting late stage seed-specific and embryo-preferential expression of a nucleic acid comprising providing the plant according to claim 7.

23. A method for altering seed properties of a plant or to produce a commercially relevant product in a plant, comprising providing the plant according to claim 7.


SEQUENCE LISTING (41 sequences)

SEQ ID NO: 1 - DNA, 6740 bp, Artificial Sequence
  T-DNA PcruP2 BnA2::GUS
aattacaacggtatatatcctgccagtactttgtggcgctctatcatagctataaacctattcagcacaatatcgattaagggccccctcgagggcgatcgctacgtacctgcaggcccgggttaattaagcggccgcaagcccaaattgtattaggtccaactaatttacaatatagaaacattataaaataagtaaacttaaaaaaaagaaaagaaaattgttcctcttcatcgggaacacaagacgatggcgaactctcaactggtgctctatactccttctgttccttatagatccattttttagaaaaaaaatttgtttcaaaaaaatatattttttacatttgtaagacattaattaataaaaaattgtacttttcaaaaaatataattgtgtttaataaaatttcattggttaaaagttattgggaatagttaattaagaaaagcaatgtattgaaaaatacaattttaaatattttttaataagcgtgaaaaaactctaacatcgatcttttaggaacggaggaagtaattatttatagttaaagtttaggttggttttctatatttttttagctgataaaacagtcataaacaaatttcaagctatgagtgtgcgtagagtgaaaatgttatagtcgtatggtttggtttgagaagattttagagataaagatagaggtcagtttttattcattgtttctatgtttttttcttgaaaacattaaaaacatagagcactctaaaccaagaatgaatcattgtatgaaagtttttttgtttgaattaattcttacattcaacaaaaagggagtgtgcctagagtatatttgtagtttataaattattttttttgttttttcgttctgatctcggtagaaaaattttctcgctcggcctctgcatgcacccgtacaaccgtcacgggcgaaacatgatgagaacacggcacgttcgtcgcgtccgcatatctttcacgtgtatctgagctgtttcgtgctgccacgtcttctctctcgagactgtattcttataatgattaccatatttgatcatttgcatgtccataaccattgatcaaagtcttacggacttgagcatttctccagttcgagtattattcgctatattacgatctagttataaaaacatattgcatagcacaaattttcttatattgtatatattcttgtctaacattataaacagaaattttgtttgtagacaactaggtactaacattagtaaaattgtcatcttaaccaagttcgatcagttctttcaagcctaattactatagtgatgtatgttgatttgttatagatcaaatgtgacatataatagaaaattattattatatcttgacaaacaaattactacaaacaataaatatatattttgtcctattatatgtgcctatctcaaagctgatgtgtaagatacatgcagttcaataagccatgcaaactgaaatgtgtcaaccctattcatcaaaatgcttttgtttaaaatgtaaagccaaactcaattttacagatgaagacataaagatagcaatttaaacgatgtctagattgtacatagtcgactctataagaacaatattttccctataaatagctataccaaaaacaagtgtaaaaccagaagaactacagcgaataagtaaagagaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcggcgcgtggcgcgcctaagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagatgaattcgatatcattaccctgttatccctaaagcttattaatataacttcgtatagcatacattatacgaagttatgtttcaaatttattatgtgttttttttccgtggtcgagattgtgtattattctttagttattacaagacttttagctaaaatttgaaagaatttactttaagaaaatcttaacatctgagataatttcagcaatagattatatttttcattactctagcagtatttttgcagatcaatcgcaacatatatggttgttagaaaaaatgcactatatatatatatattattttttcaattaaaagtgcatgatatataatatatatatatatatatatatgtgtgtgtgtatatggtcaaagaaattcttatacaaatatacacgaacacatatatttgacaaaatcaaagtattacactaaacaatgagttggtgcatggccaaaacaaatatgtagattaaaaattccagcctccaaaaaaaaatccaagtgttgtaaagcattatatatatatagtagatcccaaatttttgtacaattccacactgatcgaatttttaaagttgaatatctgacgtaggatttttttaatgtcttacctgaccatttactaataacattcatacgttttcatttgaaatatcctctataattatattgaatttggcacataataagaaacctaattggtgatttattttactagtaaatttctggtgatgggctttctactagaaagctctcggaaaatcttggaccaaatccatattccatgacttcgattgttaaccctattagttttcacaaacatactatcaatatcattgcaacggaaaaggtacaagtaaaacattcaatccgatagggaagtgatgtaggaggttgggaagacaggcccagaaagagatttatctgacttgttttgtgtatagttttcaatgttcataaaggaagatggagacttgagaagttttttttggactttgtttagctttgttgggcgttttttttttttgatcaataactttgttgggcttatgatttgtaatattttcgtggactctttagtttatttagacgtgctaactttgttgggcttatgacttgttgtaacatattgtaacagatgacttgatgtgcgactaatctttacacattaaacatagttctgttttttgaaagttcttattttcatttttatttgaatgttatatatttttctatatttataattctagtaaaaggcaaattttgcttttaaatgaaaaaaatatatattccacagtttcacctaatcttatgcatttagcagtacaaattcaaaaatttcccatttttattcatgaatcataccattatatattaactaaatccaaggtaaaaaaaaggtatgaaagctctatagtaagtaaaatataaattccccataaggaaagggccaagtccaccaggcaagtaaaatgagcaagcaccactccaccatcacacaatttcactcatagataacgataagattcatggaattatcttccacgtggcattattccagcggttcaagccgataagggtctcaacacctctccttaggcctttgtggccgttaccaagtaaaattaacctcacacatatccacactcaaaatccaacggtgtagatcctagtccacttgaatctcatgtatcctagaccctccgatcactccaaagcttgttctcattgttgttatcattatatatagatgaccaaagcactagaccaaacctcagtcacacaaagagtaaagaagaacaatggacccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgatctcacccgtctaggatcccccgatgagctaagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagataaacataacttcgtatagcatacattatacgaagttatcaaaacgtcgtgagacagtttggttaactataacggtcctaaggtagcgatcgaggcattacggcattacggcactcgcgagggtccgaatctatgtcgggtgcggagaaagaggtaatgaaatggcaattcgagcatggagccatttacaattgaatatatcctgccg

SEQ ID NO: 2 - DNA, 1543 bp, Brassica napus
aagcccaaattgtattaggtccaactaatttacaatatagaaacattataaaataagtaaacttaaaaaaaagaaaagaaaattgttcctcttcatcgggaacacaagacgatggcgaactctcaactggtgctctatactccttctgttccttatagatccattttttagaaaaaaaatttgtttcaaaaaaatatattttttacatttgtaagacattaattaataaaaaattgtacttttcaaaaaatataattgtgtttaataaaatttcattggttaaaagttattgggaatagttaattaagaaaagcaatgtattgaaaaatacaattttaaatattttttaataagcgtgaaaaaactctaacatcgatcttttaggaacggaggaagtaattatttatagttaaagtttaggttggttttctatatttttttagctgataaaacagtcataaacaaatttcaagctatgagtgtgcgtagagtgaaaatgttatagtcgtatggtttggtttgagaagattttagagataaagatagaggtcagtttttattcattgtttctatgtttttttcttgaaaacattaaaaacatagagcactctaaaccaagaatgaatcattgtatgaaagtttttttgtttgaattaattcttacattcaacaaaaagggagtgtgcctagagtatatttgtagtttataaattattttttttgttttttcgttctgatctcggtagaaaaattttctcgctcggcctctgcatgcacccgtacaaccgtcacgggcgaaacatgatgagaacacggcacgttcgtcgcgtccgcatatctttcacgtgtatctgagctgtttcgtgctgccacgtcttctctctcgagactgtattcttataatgattaccatatttgatcatttgcatgtccataaccattgatcaaagtcttacggacttgagcatttctccagttcgagtattattcgctatattacgatctagttataaaaacatattgcatagcacaaattttcttatattgtatatattcttgtctaacattataaacagaaattttgtttgtagacaactaggtactaacattagtaaaattgtcatcttaaccaagttcgatcagttctttcaagcctaattactatagtgatgtatgttgatttgttatagatcaaatgtgacatataatagaaaattattattatatcttgacaaacaaattactacaaacaataaatatatattttgtcctattatatgtgcctatctcaaagctgatgtgtaagatacatgcagttcaataagccatgcaaactgaaatgtgtcaaccctattcatcaaaatgcttttgtttaaaatgtaaagccaaactcaattttacagatgaagacataaagatagcaatttaaacgatgtctagattgtacatagtcgactctataagaacaatattttccctataaatagctataccaaaaacaagtgtaaaaccagaagaactacagcgaataagtaaagagacc

SEQ ID NO: 3 - DNA, 1500 bp, Brassica napus
ttttattgtatttgatcaactgttttttttaaatgtaaaaagacaatatttgatcaaccgtgacaaaccgtgttgaacaagggcggccaaaaaagtagcacacaaacgaattatctgtatgatatctgttcaatatttggaatatttttgctttttattattttggacctttaaatacattttttgttagcaaaaaaataataatattttgtttatataaaatcgctggaaacgtagttttgggaatgctaaatatcaaaaccataacaaataattcttttcaaaaataccacttattttattagaactaagcctggtttatctttattaaaatttaaagaaaccaaaacatagccaagaaaaattagcgatcaatctttggtaaattttattagtttggactattttctatttatttttaatttcgagcttaaattttgatttcatttgtatatatgagtttgacaaaatagtttttgtgatttagttatttactgtttattgtgtgggttggtagagaaaatgatgatttaatgaaataattaaatttgcatataaatcaaaaacagtttgttgtttaggaaaagaagtaatcgaagtgttagaaataaaaaagttacaaattcgtatttatgaaaaaaggaaaaatggaatgaaattccaaagggactgtgagcagcatgcagccgtacactcgtcatggcggtggaatatgatgaaagcacggctcgtcgcgtccgacacatctctcatgtcacgtgtgtctcagagtcatctgttttgtgtcgctacatctctcgattttcttcttatagttaccatttttgttaactttaatcatttgcatgtccatgattaatcagtcattcgaaactttaacatttctccagttcgagtgtgaccgttatactacgatctaatcaaaacatgtctagaatcaaatttacaatcttttgcatatttttttgtctaataatattactagaaatctttgtttgttactaacattagtaaaactatcatcttaaccaaattgcaggagcaattcgtttcaaacgtaattgctatagtgatgtcattgtaaatttgtcatactgatcaaatgtaaagtataagacaatttttatatatatcttgacaaacaaatcagtatatacaagaaatatagattttgtcctattacatatgcctatctcaaagttgatgtgtaaagacatgcagttcaataagccatgcaaattgagatgtgtcaaactcccttcgttaatatgtgttttcttacaacgtgaagccaaattaaattttcagaagaagacataaagatagcaactcaaatgaagtgtagattgtacatagtcgactctatatacctggttcttatctcattcaatttatcctcaaaaaactttataaacatctatataaataagttcactataaatagcttcatctaacgcagctttaaaaccagaaaaaccacaacaactaagtaaagagaaa

SEQ ID NO: 4 - DNA, 1500 bp, Brassica napus
ctctcatgtcacgtgtgtctcagagtcgttttgtgtcgctacgtcttttctctcgattgtcttcttatagttaccatttttgttaacattaatcatttgcatgtccatgattaatcagtctttcgaaactttaacatttctccgagtaggcctaggcgttcgggtacccgctggcattcagatcaggttttttggggtttttgggttttcgaattttcgggtttacgcttctaggtcccatactaaaattttattagtacggatcgggttcggttcaaaattgtattgcatcataaaacccataaagtaatcatatatcgtacggattcgggttatatcggttcggttcggatataaccaaagtaaaaaacaaattttttgaagtaaaagataaagaaaaacatctaaattaaagaaaaattaatctatcacatataaaattgataaaataacaataaaatgttaaatcaagcatgaaaacaaacatcatttataaaaaatatgtattgccttatagagagtagactttttatttcaatgagcaaattataaaatacttatttataactaattgtgtacttaaatcatttattagaattttaatatttattattatatataatattaccacaaatattgaatttaataattggaatacttatatatatttcaaaatatttatattgactattaatttcggatttttcgggttatccgttcgggttcggttaataacacttcgggttcggatattttttgtaccaccctacaagacccgttcgggtatttttacgtttcgggtcggataacggacgggttttttcagttcgggttcggttcggatttcgggttccggattttatatgcccatgcctaaattcgagtgtgaccgttaatccgttatactacgatctaatcaaaacatgtctagatcaaatttgcaatcttattgcatatttttttgtctaacaatattactagaaatctttgtttattaccaacattagtaaaactatcatcttaaccaagttgcaggagcagttcgtttcaaacgtaattgctatagtgatgttattgtaaatttgttatactgatcaaatgtaaagaataatacaatttttatatatatcttgacaaacaaatcagtatatatatacaagaaatatatattttgtcctattacatatgcctatctcaaagttgatgtgtaaagacatgcagttcaataagccatgcaaattgagatgtgtcaaactcccttcgttaatatgtgttttcttacaatgtgaagccaaattaaattttcagaagaagacataaagatagcaactcaaatgaagtgtagattgtacatagtcgactctatatacctggttcttatctcattcaatttatcctcaaaaaaatttatcaacatctatacaaataagttcactataaatagcttcatctaacgcagctgtaagaccagaaaaaccacaacaactaagtaaagagaaa

SEQ ID NO: 5 - DNA, 1500 bp, Brassica rapa
attataaaataagtaaacttaaaaaaaagaaaagaaaatttgttcctcttcatcgggaacacaagacgatggcgaactctcaactggtgctctatactccttctgttccttatagatccattttttagaaaaaaaatttgtttcaaaaaaatatattttttacatttgtaagacattaattaataaaaaattgtacttttcaaaaaatataattgtgtttaataaaatttcattggttaaaagttattgggaatagttaattaagaaaagcaatgtattgaaaaatacaattttaaatattttttaataagcgtgaaaaaactctaacatcgatcttttaggaacggaggaagtaattatttatagttaaagtttaggttggttttctatatttttttagctgataaaacagtcataaacaaatttcaagctatgagtgtgcgtagagtgaaaatgttatagtcgtatggtttggtttgagaagattttagagataaagatagaggtcagtttttattcattgtttctatgtttttttcttgaaaacattaaaaacatagagcactctaaaccaagaatgaatcattgtatgaaagtttttttgtttgaattaattcttacattcaacaaaaagggagtgtgcctagagtatatttgtagtttataaattattttttttgttttttcgttctgatctcggtagaaaaattttctcgctcggcctctgcatgcacccgtacaaccgtcacgggcgaaacatgatgagaacacggcacgttcgtcgcgtccgcatatctttcacgtgtatctgagctgtttcgtgctgccacgtcttctctctcgagactgtattcttataatgattaccatatttgatcatttgcatgtccataaccattgatcaaagtcttacggacttgagcatttctccagttcgagtattattcgctatattacgatctagttataaaaacatattgcatagcacaaattttcttatattgtatatattcttgtctaacattataaacagaaattttgtttgtagacaactaggtactaacattagtaaaattgtcatcttaaccaagttcgatcagttctttcaagcctaattactatagtgatgtatgttgatttgttatagatcaaatgtgacatataatagaaaattattattatatcttgacaaacaaattactacaaacaataaatatatattttgtcctattatatgtgcctatctcaaagctgatgtgtaagatacatgcagttcaataagccatgcaaactgaaatgtgtcaaccctattcatcaaaatgcttttgtttaaaatgtaaagccaaactcaattttacagatgaagacataaagatagcaatttaaacgatgtctagattgtacatagtcgactctataagaacaatattttccctataaatagctataccaaaaacaagtgtaaaaccagaagaactacagcgaataagtaaagagaaa

SEQ ID NO: 6 - DNA, 1500 bp, Brassica rapa
ttgctttttattattttggacctttaaatacattttttgttagttagcaaaagaaaaaatcttttgtttatgatcatatataaaatcgctagaaacgtagttttgggaatgctaaatatcaaaactataacaaataattattttcaaaaataccacttattttattagaactaagcctggtttatctttattaaaatttaaagaaaccaaaacatagccaagaaaaattagcacatgtatacgcgatttcaaatgcattaatggtagatcctttttactatattttgactatatatataatattaagttacggtatatgaataaatatataatataagttacggtatatgaaaaaaatcggagatcaatctttggtaaattttattagtttggactattttttatttatgttttaatttcgagcttaaattttgatttcgtttgtatatatgaatttgaaaagatagtttttgtgatttagttatttactgaaaaaaattaaatttgcatataaatcaaaaacagtttgttgtttaggaaaagaagtaatcgaagtgttagaaaaaaaaatggttacaaattcgtatttatgaaaaaaggaaaaatggaatgaaattccaaagggactgtgagcagcatgcagccgtacactcgtcatgggcggtggaatatgatgaaagcacggctcgtcgcgtccgacacatctctcatgtcacgtgtgtctcagagtcatctgttttgtgtcgctacgtctctcgattttcttcttatagttaccatttttgttaactttaatcatttgcatgtccatgataaatcagtctttcgaaacttttatcatttctccagttcgagtgtgaccgttatactacgatctaatcaaaacatgtctagaatcaaaaaaaaaatttttttggtgtaaatgttatctagaatcaaatttacaatcttattgcatattttttttgtctaacaatattactagaaatctttgtttgttactaacattagtaaaactatcatcttaaccaagttgcaggagcaattcgtttcaaacgtaattgctatagtgatgtcattgtaaatttgtcatactgatcaaatgtaaagtataatacaatttttatatatatcatgacaaacaaatcagtatatacaagaattatagattttgtcctattacatatgcctatctcaaagttgatgtgtaaagacatgcagttcaataagccatgcaaattgagatgtgtcaaactcccttcgttaatatgtgttttcttacaatgtgaagccaaattaaattttcagaagaagacataaagatagcaactcaaatgaagtgtagattgtacatagtcggctctatatacctggttcttatctcattcgatttatcctcaaaaaactttataaacatctatacaaataagttcactataaatagcttcatctaacgcagctgtaagaccagaaaaaccacaacaactaagtaaagagaaa

SEQ ID NO: 7 - DNA, 1500 bp, Brassica oleracea
tgctgacttctccagatactcaaatcgaaaatataaagtttggataatgatatttgtcatagatttatgtaaaagtttcaaaaagtccataaaattttaaataactaaaaatcatgttagataacttacaggttaatatatttgataattatcaataagttcatataaattcgtacatatgtaagaccatctccaatagtgctccgtatcttctctataattcccactaaaatagagtaattctattatagagttgattttgctccaatggttaactctataatagagtgaaatatagagtaatgttgttttttttcattctatatttggagtagaaaaacaacattcatctatatttcactctattatagagttgctctatttagtggaaattatagagcaccatgaagatgtcataaatgtacgtggtgtttcctttttccgatcaatttattgttagaccaaaagattaaatataatgtaattgaagtgttagaataaaaaaaaagtaattgaagtgttgttgtcaaaaagaaaaaaaaattaagtgttttaggaaaagaagtaatcgaagtgttagcagaaaaaagaaaaaaaagttacaaattcgtatttatgaaaaaaggataaatggaatgaaattccaaagggactgtgagcagcatgcagccgtacactcgtcatggcggtggaatatgatgaaagcacggctcgtcgcgtccgacacatctctcatttcacgtgtgtctcagagtcatctgttttgtgtcgctacgtcttttctctcgactgtcttcttatagttaccatttttgttaactttaatcatttgcgtgtccatgattaatcagtctttcgaaactttaacatttctccaattcgagtgtgaccgttaatccgttatactaagatctaatcaaaacatgtctagaatcaaatttgcaatcttattgcatatttttttgtctaacaatattactagaaatctttgtttgttaccaacattagtaaaactatcatcttaaccaagttgcaggagcagttcgtttcaaacgtaattgctatagtgatgttattgtaaatttgttatactgatcaaatgtaaagaataatacaatttttatatatatcttgacaaacaaatcagtatatatatacaagaaatatagattttgtcctattacatatgcctatctcaaagttgatgtgtaaagacatgcagttcaataagccatgcaaattgagatgtgtcaaactcccttcgttaatatgtgttttcttacaatgtgaagccaaattaaattttcagaagaagacataaagatagcaactcaaatgaagtgtagattgtacatagtcgactctatatacctggttcttatctcattcaatttatcctcaaaaaactttatcaacatctatacaataagttcactataaatagcttcatctaacgcagctgtaagaccagaaaaaccacaacaactaagtaaagagaaa

SEQ ID NO: 8 - DNA, 1500 bp, Brassica juncea
caagacaatggcgaactctcaactggtgctctatactccttctgttccttatagatccatttttttaaaaaaaaaaattgtttcaaaaaatatattttttgcatttgcaagacattaattaataaaaagttgtacttttcaaaaaatataattgtgtttaataaaatttcattggttaaaagttattgaaaatagttaattaagaaaaacaatgtattgaaaaatataattttaaatgttttttaataagcgtgaaaaaactataacatcgatcttttaggaacggagagagtaattatttctatttaaaagtttaggttggttttctatttttttagctgataaaacagtcataaacaaatttcaagctatgagtgtgcgtagagtgaaaatgctatagtcgtatggtttggtttgagaagattttagagataaagatagaggtcagtttttattcattgtttctatggttttttcttgaaaacattaaaaacacagagcactctaaaccaagaatgagtcattgcacgaacgtttttttgtttgaattaattcttacattcaacaaaaagggagtgtgcctagagtatatttgtaattttttgtagtttataaattattttttttgttttttcgttctgatctcggtagaaaaattttctcgctctgcctctgcatgcacccgtacaaccgtcacgggcgaaacatgatgagaacacggcacgttcgtcgcgtccgcatatctttcacgtgtatctgagctgtttcgtgctgccacgtcttctctctcgagactgtattcttataatgattaccatttttgatcatttgcatgtccataaccattgatcaaagtcttacggacttgagcatttctccagttcgagtattatcgctatactacgatctagttataaaaacatattgcatagcacaaattttcttatattgtatatattcttgtctaacattataaacagaaaatttgtttgtagacaactaggtactaacattagtaaaattgtcatcttaaccaagttcgatcagttctttcaagcctaattactatagtgatgtatgttgatttgttatagatcaaatgtgacatataatagaaaattattattatatcttgacaaacaaattactacaaacaataaatatattttttgtcctattatatgtgcctatctcaaagctgatgtgtaagagacatgcagttcaataagccatgcaaactgaaatgtgtcaaccctattcatcaaaatgcttttgtttaaaatgtaaagccaaactaaattttacagatgaagacataaagatagcaatttaaacgatgtctagattgtacatagtcgactctatatacctggttcttatctcattcaatttatcctataaattttttaatcatctataagaacaatattttccctataaatagctataccaaaaacaagtgtaaaaccagaagaactacagcgaataagtaaagagaaa

SEQ ID NO: 9 - DNA, 1500 bp, Brassica juncea
attttattgtatttgatcaactgttttttttaaatgtaaaaagacaatatttgatcaaccgtgacaaaccgtgttgaacaagggcggccaaaaaagtagcacacaaacgaattatctgtatgatatctgttcaatatttggaatatttttgctttttattattttggacctttaaatacattttttgttagcaaaaaataataatattttgtttatataaaatcgctggaaacgtagttttgggaatgctaaatatcaaaaccataacaaataattcttttcaaaaataccacttattttattagaactaagcctggtttatctttattaaaatttaaagaaaccaaaacatagccaagaaaaattagcgatcaatctttggtaaattttattagtttggactattttctatttatttttaatttcgagcttaaattttgatttcatttgtatatatgagtttgacaaaatagtttttgtgatttagttatttactgtttattgtgtgggttggtagagaaaatgatgatttaatgaaataattaaatttgcatataaatcaaaaacagtttgttgtttaggaaaagaagtaatcgaagtgttagaaataaaaaagttacaaattcgtatttatgaaaaaaggaaaaatggaatgaaattccaaagggactgtgagcagcatgcagccgtacactcgtcatggcggtggaatatgatgaaagcacggctcgtcgcgtccgacacatctctcatgtcacgtgtgtctcagagtcatctgttttgtgtcgctacatctctcgattttcttcttatagttaccatttttgttaactttaatcatttgcatgtccatgattaatcagtcattcgaaactttaacatttctccagttcgagtgtgaccgttatactacgatctaatcaaaacatgtctagaatcaaatttacaatcttttgcatatttttttgtctaataatattactagaaatctttgtttgttactaacattagtaaaactatcatcttaaccaaattgcaggagcaattcgtttcaaacgtaattgctatagtgatgtcattgtaaatttgtcatactgatcaaatgtaaagtataagacaatttttatatatatcttgacaaacaaatcagtatatacaagaaatatagattttgtcctattacatatgcctatctcaaagttgatgtgtaaagacatgcagttcaataagccatgcaaattgagatgtgtcaaactcccttcgttaatatgtgttttcttacaacgtgaagccaaattaaattttcagaagaagacataaagatagcaactcaaatgaagtgtagattgtacatagtcgactctatatacctggttcttatctcattcaatttatcctcaaaaaactttataaacatctatataaataagttcactataaatagcttcatctaacgcagctttaaaaccagaaaaaccacaacaactaagtaaagagaaa

SEQ ID NO: 10 - DNA, 1500 bp, Brassica juncea
gagatcaaaaactgacaaaagagacggattgaacaaaatattatagatcacttcttatgtttagctgattaataaaaaggtttgcacgtattacaaaacttagagacagaacaaacatttccctgtctaaattctgagatcttgcagaaaatgtcttttaaagttttttacacttaatgaaaatcattaatttttattataatcctaattaatacactattttattttatttgttaataaataagctgacaatatagataaaaatagaaaaatgatttaatatattcattgaaaatataaaacaacatttataataaaacaaattttaaagtctataaagtgaaatgagagagtacttcactttatttttcttcaaaatagaataactatataatataattaacttctaatccaatattactatattttagagtgaaaataacaaaatataaatagattattctatttatacaataaaactatttttgctatgttatacagtgagatgtgaaatagagttataacatttttattctaaactttttttataagtataaaataaaattgggttgaagatacctttagcatattcatatttatgaaaagaggaaaatggaataaaattccaaaggaactgagaagcatgcagccgtacactcgtcatgcggtggaatatgataaaagcacggctcgtcgcgtccgacacatctcttacgtcacatgtgtctcagaatcatcagttttgtgtcgccatgtctttctctcgattgtcttcttataattaccatttttgttaactttaatcatttgcatatgattaatcaagtctttcgaaactcaacatttctccaattcgagtgtgacgaccgttgtattacgatataatcaaaacatgtctagaatcaaacttgcaatcttattgcatatttttttgtctaacaaattaacaatcttactagaaatctatgtttgttattaatattagtaattctatcatcttaagagttggagcagttcgtttccgacgtaattgctatagtgatgttatcgtaaatttgttatactgatcaaatgtaaagtataatacaattttactttatataaaaactacagttttatatatatcttgacatacaaatcagtatatacaagaaatatagattttgtcctattacatacgcggatatgcctatctcaaagttgatgtgtagagacatgcagttcaataagccatgcaaattgaaatgtgtcaaactccctcgttgatatgtttttgttatagaatgtgaagacaaagtaaattttcagaagaagacataaatatagcaactcagatgaagtgtagattgtacatagtcgactctatatacctggttcttatctcattcaatttatcctctaaaaatttatcaacatatatacaaataagtccactataaatagcttcatctaacgcagctgtaagaccagaaaaacccacaacaaataagtaaagagaaa

SEQ ID NO: 11 - protein, 496 aa, Brassica napus
MARLSSLLYFSITVLIFLHGSTAQQFPNECQLDQLNALEPSHVLKAEAGRIEVWDHHAPQLRCSGVSFVRYIIESQGLYLPSFLNTAKLSFVAKGQGLMGRVVPGCAETFQDSSVFQPGSGSPFGEGQGQGQQGQGQGQGQGQGKGQQGQGKGQQGQSQGQQGQGQGFRDMHQKVEHIRSGDTIATHPGVAQWFYNNGNQPLVIVAVMDLASHQNQLDRNPRPFYLAGKNPQGQSWLHGRGQQPQNNILNGFSPEVLAQAFKIDVRTAQQLQNQQDNRGNIVRVQGPFGVIRPPLKSQRPQETEANGLEETICSARCTDNLDDPSNADVYKPQLGYISILNSYDLPILRVLRLSALRGSIRQNAMVLPQWNANANAVLYVTDGEAQIQVVNDNGDRVFDGQVSQGQLLSIPQGFSVVKRATSDQFRWIEFKTNANAQINTLAGRTSVMRGLPLEVIANGYQISLEEARRVKFNTIETTLTHSSGPASYGRPRKADA

SEQ ID NO: 12 - protein, 485 aa, Brassica napus
MARLSSLLSFSLALLIFLHGSTAQQFPNECQLDQLNALEPSHVLKAEAGRIEVWDHHAPQLRCSGVSFVRYIIESKGLYLPSFFSTAKLSFVAKGQGLMGRVVPGCAETFQDSSVFQPGGGSPFGEGQGQGQQGQGQGQQGQGQGQQGQGQQGQGQGFRDMHQKVEHIRTGDTIATHPGVAQWFYNDGNQPLVIVSVLDLASHQNQLDRNPRPFYLAGNNPQGQVWIEGREKQPQKNILNGFTPEVLAKAFKIDVRTAQQLQNQEDNRGNIIRVQGPFSVIRPPLRSQRPQEEVNGLEETICSARCTDNLDDPSNADVYKPQLGYISTLNSYDLPILRFLRLSALRGSIRQNAMVLPQWNANANAVLYVTDGEAHVQVVNDNGDRVFDGQVSQGQLLSIPQGFSVVKRATSEQFRWIEFKTNANAQINTLAGRTSVLRGLPLEVISNGYQISLEEARRVKFNTIETTLTHSSGPASYGRPMKADA

SEQ ID NO: 13 - protein, 489 aa, Brassica napus
MARLSSLLSFSLALLIFLHGSTAQQFPNECQLDQLNALEPSHVLKAEAGRIEVWDHHAPQLRCSGVSFVRYIIESKGLYLPSFFSTAKLSFVAKGEGLMGRVVPGCAETFQDSSVFQPGGGSPFGEGQGQGQQGQGQGHQGQGQGQQGQQGQQGQQSQGQGFRDMHQKVEHIRTGDTIATHPGVAQWFYNDGNQPLVIVSVLDLASHQNQLDRNPRPFYLAGNNPQGQVWIEGREQQPQKNILNGFTPEVLAKAFKIDVRTAQQLQNQQDNRGNIIRVQGPFSVIRPPLRSQRPQEEVNGLEETICSARCTDNLDDPSNADVYKPQLGYISTLNSYDLPILRFLRLSALRGSIRQNAMVLPQWNANANAVLYVTDGEAHVQVVNDNGDRVFDGQVSQGQLLSIPQGFSVVKRATSEQFRWIEFKTNANAQINTLAGRTSVLRGLPLEVISNGYQISLEEARRVKFNTIETTLTHSSGPASYGGPRKADA

SEQ ID NO: 14 - protein, 496 aa, Brassica rapa; identical to SEQ ID NO: 11

SEQ ID NO: 15 - protein, 485 aa, Brassica rapa
MARLSSLLSFSLALLIFLHGSTAQQFPNECQLDQLNALEPSHVLKAEAGRIEVWDHHAPQLRCSGVSFVRYIIESKGLYLPSFFSTAKLSFVAKGQGLMGRVVPGCAETFQDSSVFQPGGGSPFGEGQGQGQQGQGQGQQGQGQGQQGQGQQGQGQGFRDMHQKVEHIRTGDTIATHPGVAQWFYNDGNQPLVIVSVLDLASHQNQLDRNPRPFYLAGNNPQGQVWIEGREQQPQKNILNGFTPEVLAKAFKIDVRTAQQLQNQQDNRGNIVRVQGPFSVIRPPLRSQRPQEEVNGLEETICSARCTDNLDDPSNADVYKPQLGYISTLNSYDLPILRFLRLSALRGSIRQNAMVLPQWNANANAVLYVTDGEAHVQVVNDNGDRVFDGQVSQGQLLSIPQGFSVVKRATSEQFRWIEFKTNANAQINTLAGRTSVLRGLPLEVISNGYQISLEEARRVKFNTIETTLTHSSGPASYGRPRKADA

SEQ ID NO: 16 - protein, 489 aa, Brassica oleracea
MARLSSLLSFSLALLIFLHGSTAQQFPNECQLDQLNALEPSHVLKAEAGRIEVWDHHAPQLRCSGVSFVRYIIESKGLYLPSFFSTAKLSFVAKGQGLMGRVVPGCAETFQDSSVFQPGGGSPFGEGQGQGQQGQGQGHQGQGQGQQGQQGQQGQQSQGQGFRDMHQKVEHIRTGDTIATHPGVAQWFYNDGNQPLVIVSVLDLASHQNQLDRNPRPFYLAGNNPQGQVWIEGREQQPQKNILNGFTPEVLAKAFKIDVRTAQQLQNQQDNRGNIIRVQGPFSVIRPPLRSQRPQEEVNGLEETICSARCTDNLDDPSNADVYKPQLGYISTLNSYDLPILRFLRLSALRGSIRQNAMVLPQWNANANAVLYVTDGEAHVQVVNDNGDRVFDGQVSQGQLLSIPQGFSVVKRATSEQFRWIEFKTNANAQINTLAGRTSVLRGLPLEVISNGYQISLEEARRVKFNTIETTLTHSSGPASYGGPRKADA

SEQ ID NO: 17 - protein, 437 aa, Brassica juncea
MARLSSLLYFSITVLIFLHGSTAQQFPNECQLDQLNALEPSHVLKAEAGRIEVWDHHAPQLRCSGVSFVRYIIESQGLYLPSFLNTAKLSFVAKGQGLMGRVVPGCAETFQDSSVFQPGSGSPFGEGQGQGQQGQGQGQGQGKGQQGQSQGQQGQGQGFRDMHQKVEHIRSGDTIATHPGVAQWFYNNGNQPLVIVAVMDLASHQNQLDRNPRNQQDNRGNIVRVQGPFGVIRPPLKSQRPQETEANGLEETICSARCTDNLDDPSNADVYKPQLGYISILNSYDLPILRVLRLSALRGSIRQNAMVLPQWNANANAVLYVTDGEAQIQVVNDNGDRVFDGQVSQGQLLSIPQGFSVVKRATSDQFRWIEFKTNANAQINTLAGRTSVMRGLPLEVIANGYQISLEEARRVKFNTIETTLTHSSGPASYGRPRKADA

SEQ ID NO: 18 - protein, 485 aa, Brassica juncea
MARLSSLLSFSLALLIFLHGSTAQQFPNECQLDQLNALEPSHVLKAEAGRIEVWDHHAPQLRCSGVSFVRYIIESKGLYLPSFFSTAKLSFVAKGQGLMGRVVPGCAETFQDSSVFQPGGGSPFGEGQGQGQQGQGQGQQGQGQGQQGQGQQGQGQGFRDMHQKVEHIRTGDTIATHPGVAQWFYNDGNQPLVIVSVLDLASHQNQLDRNPRPFYLAGNNPQGQVWIEGREKQPQKNILNGFTPEVLAKAFKIDVRTAQQLQNQQDNRGNIVRVQGPFSVIRPPLRSQRPQEEVNGLEETICSARCTDNLDDPSNADVYKPQLGYISTLNSYDLPILRFLRLSALRGSIRQNAMVLPQWNANANAVLYVTDGEAHVQVVDDNGDRVFDGQVSQGQLLSIPQGFSVVKRATSEQFRWIEFKTNANAQINTLAGRTSVLRGLPLEVISNGYQISLEEARRVKFNTIETTLTHSSGPASYGRPRKADA

SEQ ID NO: 19 - protein, 473 aa, Brassica juncea
MARFSSLLSFSLALLIFLHGSTAQQFPNECQLDQLNALEPSYVLKAEAGRIEVWDHHAPQLRCSGVSFVRYIIESKGLYLPSFFSTAKLSFVAKGQGLMGRVVPGCAETFQDSPVFQPGGGSPFGGQQGQQGQQGQGQQGQQGQGFRDMHQKVEHIRTGDTIATHPGVAQWFYNDGNKPLVIVSVLDLASNQNQLDRNPRPFYLAGNNPQGQVWLQGREQQPQKNILNGFTPEVIAQAFKINVRTAQQLQNQQDNRGNIVRVQGPFSVIRPPLRSQRPQEEVNGLEETICSARCTDNLDDPSNADVYKPQLGYISTLNSYDLPILRVLRLSALRGSIHQNAMVLPQWNANANAVLYVTDGEAHVQVVNDNGDRVFDGQLSQGQLLAIPQGFSVVKRATSEQFRWIEFKTNANAQINTLAGRTSVLRGLPLEVISNGYQISLEEARRVKFNTIETTLTHSSGPASYGRPRKADA

SEQ ID NO: 20 - DNA, 1491 bp, Brassica napus
atggctcgactctcgtctcttctctatttttcgataacagttttgatctttctccatggctctacagctcaacagtttccaaacgagtgccaactagaccagctcaatgcgctggagccgtcacacgtacttaaggccgaggctggtcgcatcgaagtgtgggaccaccacgctcctcagctacgctgctctggtgtctccttcgtacgttacataatcgagtctcagggtctatacttgccctctttcttaaataccgcgaagctctctttcgttgctaaaggacaaggccttatggggagagtggtccctggatgcgctgagactttccaggactcatcagtattccaaccaggcagtggcagccccttcggagaaggtcaaggccaaggtcagcagggtcaggggcaaggtcagggtcagggtcaaggcaagggccaacagggtcaaggcaagggccaacagggtcaatcccagggccaacagggtcaaggtcaaggtttccgtgatatgcaccagaaagtagagcacataaggagcggcgacaccattgctacacatcccggtgtagctcaatggttctacaacaatggaaaccaacctcttgtcatcgttgccgtcatggatttagctagccaccagaaccagcttgaccgcaacccaaggccattttacttagcaggaaaaaacccacaaggccaatcatggctacacggacgagggcaacagccacaaaacaacatccttaatggcttctctccagaggttcttgctcaagcgttcaagatcgatgttaggacagcgcaacaacttcagaaccagcaagataaccggggaaacattgtccgtgtccaaggccccttcggtgttattaggccgccattgaaaagccagagaccacaggagacagaagctaacggtctagaagagaccatatgcagcgcaaggtgcacggataacctcgatgacccatctaacgcggatgtgtataagccacagcttggttacatcagcattcttaacagttatgatctacccatccttcgcgtacttcgcctctcagccctccgtggatcaatccgtcaaaatgcaatggttcttccacagtggaatgcaaatgcaaacgcggttctctacgtgacagacggggaagcccaaatacaggtggttaacgacaacggtgacagagtgttcgatggacaagtctctcaagggcagctactttccattccacaaggattctccgttgtgaaacgcgcaacaagcgatcagttcaggtggatagaattcaagacaaacgcaaacgcccagatcaacactcttgctggacgtacctcagtcatgagaggtttaccattagaggtcatagccaatgggtaccaaatctcacttgaagaagcaagaagggttaagttcaacacaatagagaccactttgacccacagtagtggcccagcgagctacggaaggccaaggaaggctgatgcttga

SEQ ID NO: 21 - DNA, 1458 bp, Brassica napus
atggctcggctctcatctcttctctctttttccttagcacttttgatctttctccatggctctacagctcaacagtttccaaacgagtgtcagctagaccagctcaatgcactggagccgtcacacgtacttaaggctgaggctggtcgcatcgaggtgtgggaccaccacgctcctcagctacgttgctctggtgtctccttcgtacgttacatcatcgagtctaagggtctctacttgccctctttctttagcaccgcgaagctctccttcgtggctaaaggacaaggtcttatggggagagtagtccctggatgcgccgagacattccaggactcatcagtgttccaaccaggcggtggtagccccttcggagaaggccagggccagggacaacaaggtcagggccaaggccagcagggtcaaggccagggacaacagggccagggccaacagggtcaaggccagggcttccgtgatatgcaccagaaagtggagcacataaggaccggggacaccatcgctacacatcccggtgtagcccaatggttctacaacgatggaaaccaaccacttgttatcgtttccgtcctcgatttagccagccaccagaaccagctcgaccgcaacccaaggccattttacttagccggaaacaacccacaaggccaagtatggatagaaggacgagagaaacagccacaaaagaacatccttaatggcttcacaccagaagttcttgctaaagcttttaagatagatgttaggacagcgcagcaacttcagaaccaggaagacaaccgtggaaacattatccgagtccaaggcccattcagtgtcattaggccgcctttgaggagtcagagaccgcaggaggaagttaacggtttagaagagaccatatgcagcgcgaggtgcaccgataacctcgatgatccatctaatgctgacgtatacaagccacagctcggttacatcagcactctcaacagctacgatctccccatccttcgcttccttcgtctctcagccctccgtggatctattcgtcaaaacgcgatggtgcttccacagtggaacgcaaacgcaaacgcggttctctacgtgacagacggggaagcccatgtgcaggtggttaacgacaacggtgacagagtgttcgacggacaagtctctcaaggacagctactttccataccacaaggtttctccgtggtgaaacgcgcaacaagcgaacagttccggtggatcgagttcaagacaaacgcaaacgcacagatcaacacacttgctggacgaacctcggtcttgagaggtttaccattagaggtcatatccaatgggtaccaaatctcactcgaagaagcaagaagggttaagttcaacacgatcgagaccactttgacgcacagcagtggcccagctagctacggaaggccaatgaaggctgatgcttaa

SEQ ID NO: 22 - DNA, 1470 bp, Brassica napus
atggctcggctctcatctcttctctctttttccttagcacttttgatctttctccatggctctacagctcaacagtttccaaacgagtgtcagctagaccagctcaatgcactggagccgtcacacgtacttaaggctgaggctggtcgcatcgaggtgtgggaccaccacgctcctcagctacgttgctctggtgtctcctttgtacgttacatcatcgagtctaagggtctctacttgccctctttctttagcaccgcgaagctctccttcgtggctaaaggagaaggtcttatggggagagtggtccctggatgcgccgagacattccaggactcatcagtgtttcaaccaggcggtggtagccccttcggagaaggtcagggccagggacaacaaggtcagggccaaggccaccagggtcaaggccagggacaacagggccaacaaggtcagcaaggacaacagagtcaaggccagggcttccgtgatatgcaccagaaagtggagcacataaggactggggacaccatcgctacacatcccggtgtagcccaatggttctacaacgacggaaaccaaccacttgtcatcgtttccgtcctcgatttagccagccaccagaatcagctcgaccgcaacccaaggccattttacttagccggaaacaacccacaaggccaagtatggatagaaggacgcgagcaacagccacaaaagaacatccttaatggcttcacaccagaggttcttgctaaagctttcaagatcgatgttaggacagcgcaacaacttcagaaccagcaagacaaccgtggaaacattatccgagtccaaggcccattcagtgtcattaggccgcctttgaggagtcagagaccgcaggaggaagttaacggtttagaagagaccatatgcagcgcgaggtgcaccgataacctcgatgatccatctaatgctgacgtatacaagccacagctcggttacatcagcactctgaacagctatgatctccccatccttcgcttccttcgtctctcagccctccgtggatctatccgtcaaaacgcgatggtgcttccacagtggaacgcaaacgcaaacgcggttctctacgtgacagacggggaagcccatgtgcaggtggttaacgacaacggtgacagagtgttcgacggacaagtctctcaaggacagctactttccataccacaaggtttctccgtggtgaaacgcgcaacaagcgaacagttccggtggatcgagttcaagacaaacgcaaacgcacagatcaacacacttgctggacgaacctcggtcttgagaggtttaccattagaggtcatatccaatgggtaccaaatctcactcgaagaagcaagaagggttaagttcaacacgatcgagaccactttgacgcacagcagtggcccagctagctacggagggccaaggaaggctgatgcttaa

SEQ ID NO: 23 - DNA, 1491 bp, Brassica rapa; identical to SEQ ID NO: 20

SEQ ID NO: 24 - DNA, 1458 bp, Brassica rapa
atggctcggctctcatctcttctctctttttccttagcacttttgatctttctccatggctctacagctcaacagtttccaaacgagtgtcagctagaccagctcaatgcactggagccgtcacacgtacttaaggctgaggctggtcgcatcgaggtgtgggaccaccacgctcctcagctacgttgctctggtgtctccttcgtacgttacatcatcgagtctaagggtctctacttgccctctttctttagcaccgcgaagctctccttcgtggctaaaggacaaggtcttatggggagagtagtccctggatgcgccgagacattccaggactcatcagtgttccaaccaggcggtggtagccccttcggagaaggccagggccagggccaacaaggtcagggccaaggccagcagggtcaaggccagggacaacagggccagggccaacagggtcaaggccagggcttccgtgatatgcaccagaaagtggagcacataaggaccggggacaccatcgctacacatcccggtgtagcccaatggttctacaacgatggaaaccaaccacttgttatcgtttccgtcctcgatttagccagccaccagaaccagctcgaccgcaacccaaggccattttacttagccggaaacaacccacaaggccaagtatggatagaaggacgagagcaacagccacaaaagaacatccttaatggcttcacaccagaagttcttgctaaagcttttaagatagatgttaggacagcgcaacaacttcagaaccagcaagacaaccgtggaaacatcgtccgagtccaaggcccattcagtgtcattaggccgcctttgaggagtcagagaccgcaggaggaagttaacggtttagaagagaccatatgcagcgcgaggtgcaccgataacctcgatgatccatctaatgctgacgtatacaagccacagctcggttacatcagcactctcaacagctacgatctccccatccttcgcttccttcgtctctcagccctccgtggatctattcgtcaaaacgcgatggtgcttccacagtggaacgcaaacgcaaacgcggttctctacgtgacagacggggaagcccatgtgcaggtggttaacgacaacggtgacagagtgttcgacggacaagtctctcaaggacagctactttccataccacaaggtttctccgtggtgaaacgcgcaacaagcgaacagttccggtggatcgagttcaagacaaacgcaaacgcacagatcaacacacttgctggacgaacctcggtcttgagaggtttaccattagaggtcatatccaatgggtaccaaatctcactcgaagaagcaaggagggttaagttcaacacgatcgagaccactttgacgcacagcagtggcccagctagctacggaaggccaaggaaggctgatgcttaa

SEQ ID NO: 25 - DNA, 1470 bp, Brassica oleracea
atggctcggctctcatctcttctctctttttccttagcacttttgatctttctccatggctctacagctcaacagtttcctaacgagtgtcagctagaccagctcaatgcactggagccgtcacacgtacttaaggctgaggctggtcgcatcgaggtgtgggaccaccacgctcctcagctacgttgctctggtgtctcctttgtacgttacatcatcgagtctaagggtctctacttgccctctttctttagcaccgcgaagctctccttcgtggctaaaggacaaggtcttatggggagagtggtccctggatgcgccgagacattccaggactcatcagtgttccaaccaggcggtggtagccccttcggagaaggtcagggccagggacaacaaggtcagggccaaggccaccagggtcaaggccagggacaacagggccaacaaggtcagcaaggacaacagagtcaaggccagggcttccgtgatatgcaccagaaagtggagcacataaggactggggacaccatcgctacacatcccggtgtagcccaatggttctacaacgatggaaaccaaccacttgtcatcgtttccgtcctcgatttagccagccaccagaaccagctcgaccgcaacccaaggccattttacttagccggaaacaacccacaaggccaagtatggatagaaggacgcgagcaacagccacaaaagaacatccttaatggcttcacaccagaggttcttgctaaagctttcaagatcgatgttaggacagcgcaacaacttcagaaccagcaagacaaccgtggaaacattatccgagtccaaggcccattcagtgtcattaggccgcctttgaggagtcagagaccgcaggaggaagttaacggtttagaagagaccatatgcagcgcgaggtgcaccgataacctcgatgacccatctaatgctgacgtatacaagccacagctcggttacatcagcactctgaacagctatgatctccccatccttcgcttccttcgtctctcagccctccgtggatctatccgtcaaaacgcgatggtgcttccacagtggaacgcaaacgcaaacgcggttctctacgtgacagacggggaagcccatgtgcaggtggttaacgacaacggtgacagagtgttcgacggacaagtctctcaaggacagctactttccataccacaaggtttctccgtggtgaaacgcgcaacaagcgaacagttccggtggatcgagttcaagacaaacgcaaacgcacagatcaacacacttgctggacgaacctcggtcttgagaggtttaccattagaggtcatatccaatgggtaccaaatctcactcgaagaagcaagaagggttaagttcaacacgatcgagaccactttgacgcacagcagtggcccagctagctacggagggccaaggaaggctgatgcttaa

SEQ ID NO: 26 - DNA, 1314 bp, Brassica juncea
atggctcgactctcgtctcttctctatttttcgataacagttttgatctttctccatggctctacagctcaacagtttccaaacgagtgccaactagaccagctcaatgcgctggagccgtcacacgtacttaaggccgaggctggtcgcatcgaagtgtgggaccaccacgctcctcagctacgctgctctggtgtctccttcgtacgttacataatcgagtctcagggtctatacttgccctctttcttaaataccgcgaagctctctttcgttgctaaaggacaaggccttatggggagagtggtccctggatgcgctgagactttccaggactcatcagtattccaaccaggcagtggcagccccttcggagaaggtcaaggccaaggtcagcagggtcaggggcaaggtcagggtcaaggcaagggccaacagggtcaatcccagggccaacagggtcaaggtcaaggtttccgtgatatgcaccagaaagtagagcacataaggagcggcgacaccattgctacacatcccggtgtagctcaatggttctacaacaatggaaaccaacctcttgtcatcgttgccgtcatggatttagctagccaccagaaccagcttgaccgcaacccaaggaaccagcaagacaaccggggaaacattgtccgtgtccaaggccccttcggtgttattaggccgccattgaaaagccagagaccacaggagacagaagctaacggtctagaagagaccatatgcagcgcaaggtgcacggataacctcgatgacccatctaacgcggatgtgtataagccacagcttggttacatcagcattcttaacagttatgatctacccatccttcgcgtacttcgcctctcagccctccgtggatcaatccgtcaaaatgcaatggttcttccacagtggaatgcaaatgcaaacgcggttctctacgtgacagacggggaagcccaaatacaggtggttaacgacaacggtgacagagtgttcgatggacaagtctctcaagggcagctactttccattccacaaggattctccgttgtgaaacgcgcaacaagcgatcagttcaggtggatagaattcaagacaaacgcaaacgcccagatcaacactcttgctggacgtacctcagtcatgagaggtttaccattagaggtcatagccaatgggtaccaaatctcacttgaagaagcaagaagggttaagttcaacacaatagagaccactttgacccacagtagtggcccagcgagctacggaaggccaaggaaggctgatgcttga

SEQ ID NO: 27 - DNA, 1458 bp, Brassica juncea
atggctcggctctcatctcttctctctttttccttagcacttttgatctttctccatggctctacagctcaacagtttccaaacgagtgtcagctagaccagctcaatgcactggagccgtcacacgtacttaaggctgaggctggtcgcatcgaggtgtgggaccaccacgctcctcagctacgttgctctggtgtctccttcgtacgttacatcatcgagtctaagggtctctacttgccctctttctttagcaccgcgaagctctccttcgtggctaaaggacaaggtcttatggggagagtagtccctggatgcgccgagacattccaggactcatcagtgttccaaccaggcggtggtagccccttcggagaaggccagggccagggacaacaaggtcagggccaaggccagcagggtcaaggccagggacaacagggccagggccaacagggtcaaggccagggcttccgtgatatgcaccagaaagtggagcacataaggaccggggacaccatcgctacacatcccggtgtagcccaatggttctacaacgatggaaaccaaccacttgttatcgtttccgtcctcgatttagccagccaccagaaccagctcgaccgcaacccaaggccattttacttagccggaaacaacccacaaggccaagtatggatagaaggacgagagaaacagccacaaaagaacatccttaatggcttcacaccagaagttcttgctaaagcttttaagatagatgttaggacagcgcaacaacttcagaaccagcaagacaaccgtggaaacatcgtccgagtccaaggcccattcagtgtcattaggccgcctttgaggagtcagagaccgcaggaggaagttaacggtttagaagagaccatatgcagcgcgaggtgcaccgataacctcgatgatccatctaatgctgacgtatacaagccacagctcggttacatcagcactctcaacagctacgatctccccatccttcgcttccttcgtctctcagccctccgtggatctattcgtcaaaacgcgatggtgcttccacagtggaacgcaaacgcaaacgcggttctctacgtgacagacggggaagcccatgtgcaggtggttgacgacaacggtgacagagtgttcgacggacaagtctctcaaggacagctactttccataccacaaggtttctccgtggtgaaacgcgcaacaagcgaacagttccggtggatcgagttcaagacaaacgcaaacgcacagatcaacacacttgctggacgaacctcggtcttgagaggtttaccattagaggtcatatccaatgggtaccaaatctcactcgaagaagcaagaagggttaagttcaacacgatcgagaccactttgacccacagcagtggcccagctagctacggcaggccaaggaaggctgatgcttaa

SEQ ID NO: 28 - DNA, 1422 bp, Brassica juncea
atggctcggttctcatctcttctctctttttccttagcacttttgatctttctccatggctcaacagctcagcagtttccaaacgagtgccagctagaccagctgaatgcgctcgagccgtcatacgtacttaaggccgaggctggtcgcatcgaggtgtgggaccaccacgctcctcagctacgttgctctggtgtctccttcgtacgttacatcatcgagtctaagggtctctacttgccttctttctttagcaccgcgaagctctccttcgtggctaaaggacaaggtcttatggggagagttgtccctggatgcgccgagacattccaggactcaccagtgttccaaccaggcggtggcagccctttcggaggccagcagggacagcagggacaacagggccagggacagcagggacaacagggccagggcttccgtgatatgcaccagaaagtggagcatataaggaccggcgacacgattgctacacatcccggtgtagcccaatggttctacaacgatggaaacaaacccctagtcatcgtttccgtccttgatttagctagcaaccagaaccagctggaccgcaacccaaggccattttacttagccggaaacaacccacaaggccaagtatggctacaaggacgagagcaacagccacaaaagaacatccttaatggcttcacaccagaggttattgctcaagctttcaagatcaatgttaggacagcgcaacaacttcagaaccagcaagacaaccgtggaaacattgtccgagtccagggcccattcagtgtcattaggccgcctttgaggagtcagagaccgcaggaggaagttaacggtttagaagagaccatatgcagcgcgaggtgcaccgataacctcgatgacccatctaatgctgacgtgtacaagccacagctcggttacatcagcactcttaacagctacgatctccccatccttcgcgtacttcgtctctcagccctccgtggatctattcatcaaaacgcaatggtgcttccacagtggaacgcaaatgcaaacgcggttctctacgtgacagacggggaagcacatgtgcaggtggttaacgacaacggtgacagagtgttcgacggacaactctctcaaggacagctacttgccataccacaaggtttctccgtggtgaaacgcgcaacaagcgaacagttccgctggatcgagttcaagacaaacgcaaacgcgcagatcaacacacttgctggacgcacctcggtcttgagaggtttacccttagaggtcatatccaatgggtaccaaatctcactcgaggaagcaagaagggtcaagttcaacacgatcgagaccactttgacacacagcagtggcccagctagctacggaaggccaaggaaagctgatgcttga

SEQ ID NO: 29 - DNA, 16 bp, Artificial Sequence
  motif 1
twttgyatatwttytt

SEQ ID NO: 30 - DNA, 14 bp, Artificial Sequence
  motif 3
aytaayattagtaa

SEQ ID NO: 31 - DNA, 20 bp, Artificial Sequence
  motif 6
cstaattrctatagtgatgt

SEQ ID NO: 32 - DNA, 10 bp, Artificial Sequence
  motif 7
atttgtyata

SEQ ID NO: 33 - DNA, 10 bp, Artificial Sequence
  motif 8
gatcaaatgt

SEQ ID NO: 34 - DNA, 28 bp, Artificial Sequence
  motif 9
tatatcwtgacawacaaatyastayawa

SEQ ID NO: 35 - DNA, 29 bp, Artificial Sequence
  motif 10
caakaawtatakwttttgtcctattayat

SEQ ID NO: 36 - DNA, 24 bp, Artificial Sequence
  motif 11
tgcctatctcaaagytgatgtgta

SEQ ID NO: 37 - DNA, 40 bp, Artificial Sequence
  motif 12
acatgcagttcaataagccatgcaaaytgaratgtgtcaa

SEQ ID NO: 38 - DNA, 24 bp, Artificial Sequence
  motif 13
cagawgaagacataaakatagcaa

SEQ ID NO: 39 - DNA, 25 bp, Artificial Sequence
  motif 14
tagattgtacatagtcgrctctata

SEQ ID NO: 40 - DNA, 15 bp, Artificial Sequence
  motif 15
taaraccagaaraac

SEQ ID NO: 41 - DNA, 12 bp, Artificial Sequence
  motif 16
taagtaaagaga